(19) United States
(12) Patent Application Publication
(10) Pub. No.: US 2005/0191317 A1
(43) Pub. Date: Sep. 1, 2005

(54) Ghrelin-carrier conjugates

(75) Inventors: Martin F. Bachmann, Seuzach (CH); Alma Fulurija, Zurich-Schlieren (CH)

Correspondence Address: STERNE, KESSLER, GOLDSTEIN & FOX PLLC, 1100 NEW YORK AVENUE, N.W., WASHINGTON, DC, 20005, US

(73) Assignee: Cytos Biotechnology AG, Zurich-Schlieren (CH)

(21) Appl. No.: 11/037,396

(22) Filed: Jan. 19, 2005

Related U.S. Application Data
(60) Provisional application No. 60/537,230, filed on Jan. 20, 2004.

(51) Int. Cl. A61K 39/12; C12N 15/861; C12N 7/00

(52) U.S. Cl. 424/199.1; 435/456; 435/235.1

(57) ABSTRACT

The present invention provides ordered and repetitive antigen arrays comprising, inter alia, compositions comprising a virus-like particle (VLP) to which is linked at least one antigen, wherein said antigen is grehlin or peptides or fragments thereof. The invention also provides methods for producing the aforesaid compositions. The compositions and methods of the invention are useful in the production of vaccines and to efficiently induce self-specific immune responses, in particular antibody responses. The invention also provides for compositions and methods for the prevention and/or treatment of ghrelin-related conditions, disorders or diseases. For example, the compositions of the invention are useful in the production of vaccines for the prevention or treatment of obesity and other disease associated with increased food-uptake or increased body weight.

### CROSS REFERENCE TO RELATED APPLICATIONS

This application claims the benefit of U.S. Provisional Application No. 60/537,230, filed Jan. 20, 2004, which is entirely incorporated herein by reference.

### BACKGROUND OF THE INVENTION

1. Field of the Invention

The present invention is related to the fields of molecular biology, virology, immunology and medicine. The invention provides a modified virus-like particle (VLP) comprising a VLP and particular peptides derived from ghrelin linked thereto.

The invention also provides a process for producing the modified VLP. The modified VLPs of the invention are useful in the production of vaccines for the treatment of obesity and other disease associated with increased food-uptake or increased body weight and to efficiently induce immune responses, in particular antibody responses. Furthermore, the compositions of the invention are particularly useful to efficiently induce self-specific immune responses within the indicated context.

2. Related Art

Obesity is a disease afflicting millions of people world-wide. Many factors regulate hunger and feeding behaviour, including leptin, growth-hormone (GH), neuropeptide Y (NPY), agouti-related protein (AGRP) and others. A recently identified key regulator of feeding behaviour is ghrelin, an acylated peptide produced in the stomach and also some parts of the brain (hypothalamus) (Kojima et al., Nature 402:656-660 (1999)). Ghrelin is derived by encymatic cleavage from a prepro form encompassing 117 amino acids resulting in a 28 amino acid long peptide with a n-octanoylation at serine 3. Biologically active ghrelin needs to be n-octanyolated at this position. A second, 27 aa isoform of ghrelin (Ghrelin-desQ14), lacking a glutamine (Q) at position 14, has been identified, however, this isoform represents only a minor component of circulating ghrelin. Like full length ghrelin, the biological activity of Ghrelin-des-Q14 is dependant on the n-octanoyl group on serine 3. Nevertheless, most functional activity is derived from the 28 aa ghrelin isoform (Hosoda et al., Biochem. Biophys. Res. Commun. 279(3):909-913 (2000)). Ghrelin is highly conserved, since human and rat ghrelin differ by only 2 amino-acids.

Receptors for ghrelin (GHS-R) are expressed in various regions of the brain, including the arcuate nucleus (Arc) and ventromedial nucleus of the hypothalamus and in the pituitary gland (Howard et al., Science 273:974-977 (1996)); McKee et al., Mol Endokrin. 11:415-423 (1997); Guan et al., Mol Brain Research 48:23-29 (1997)), indicating that ghrelin primarily acts in the brain. In addition to stimulating release of GH from the pituitary gland (Kojima et al., Nature 402:656-660 (1999)), ghrelin has more recently been identified as a key central regulator of feeding (Nakazato et al., Nature 409:194-198 (2001)). Specifically, upon intracerebroventricular application, ghrelin was shown to stimulate feeding. Moreover, intracerebroventricular application of anti-ghrelin antibodies inhibited feeding. Ghrelin injection induced upregulated release of NPY and anti-NPY antibodies together with AGRP antagonists blocked ghrelin induced feeding, suggesting that ghrelin modulates feeding via enhancing expression of NPY and AGRP (Nakazato et al., Nature 409:194-198 (2001)). Moreover, peripheral daily administration of ghrelin induced body weight gain in mice and rats and serum ghrelin concentrations were increased in fasting rats and reduced by feeding, further suggesting that ghrelin plays a key role in regulating feeding (Tschop et al., Nature 407:908-912). Transgenic rats expressing anti-sense GHS-R RNA in the Arc exhibited lower body weight and less adipose tissue, supporting the notion that ghrelin regulates body weight (Shuto et al., JCI 109:14291436 (2002)). There is also evidence for a key role for ghrelin in human feeding behaviour. Peripheral administration of ghrelin in humans enhanced appetite and increased food uptake in humans (Wren et al., J Clin Endocrinol Metab 86:5992-5998 (2001)). Humans with Prader-Willi syndrome, the most common form of human syndromic obesity, exhibit highly increased ghrelin levels (Cummings et al., Nat Med 8:643-644 (2002)). In addition, plasma ghrelin levels in humans are strongly increased after diet-induced weight loss, correlating with rapid regain of weight when people stop the diet. In contrast, in patients with gastric bypass surgery, ghrelin levels remained low during and after diet and patients do not usually regain their weight under these conditions ((Cummings et al., N Engl J Med 21:1623-1630 (2002)). Hence, ghrelin appears to be a key regulator of food uptake and body weight in humans.

Since peripheral administration of ghrelin was able to increase food uptake leading to increased body weight (Tschop et al., Nature 407:908-912), it is likely that ghrelin produced in the stomach reaches the brain through the blood stream and triggers feeding. Thus, it may be possible to block migration of ghrelin from the blood to the brain to stop food uptake in animals and humans. As it has been shown that specific antibodies can block ghrelin action in the brain (Nakazato et al., Nature 409:194-198 (2001)) it is likely that peripheral antibodies will also be able to block the action of peripheral ghrelin. In addition, since antibodies inefficiently penetrate the blood brain barrier, ghrelin-specific antibodies would probably be able to seclude ghrelin from the brain but would not act on ghrelin within the brain. This would be a particularly attractive possibility, since ghrelin is also produced in the brain where it probably exerts functions different from regulating food uptake (Nakazato et al., Nature 409:194-198 (2001)). Therefore, a potential therapy for obesity would be to induce ghrelin-specific antibodies in the host, leading to the long-term blockage obstruction of ghrelin resulting in reduced food-uptkake, similarly to that observed in gastric bypass patients.

WO 98/42840 discloses the influence of ghrelin and ghrelin-derived fragments on the gastrointestinal tract and hereby in particular their effect on gastric motility and gastric emptying. Moreover, U.S. Pat. No. 6,420,521 discloses the use of short ghrelin peptides for effects on gastric function, including gastric emptying, gastric contractility and glucose absorption.

WO 02/056905 discloses a composition comprising an ordered and repetitive antigen or antigenic determinant array. The ordered and repetitive antigen or antigenic determinant is useful in the production of vaccines for the treatment of infectious diseases, the treatment of allergies and as a pharmaccine to prevent or cure cancer and to efficiently induce self-specific immune responses, in particular antibody responses.

### BRIEF SUMMARY OF THE INVENTION

We have found that particular ghrelin-peptides, which are bound to a core particle having a structure with an inherent repetitive organization, and hereby in particular to virus-like-particles (VLPs) and subunits of VLPs, respectively, particularly when leading to highly ordered and repetitive conjugates, represent potent immunogens for the induction of specific antibodies. We found that short peptides derived from the N-terminus of ghrelin such as 1-6,1-7 or 1-8, and hereby in particular 1-8, and coupled to VLPs were able to induce strong antibody responses against the native form of ghrelin. This was suprising, since native ghrelin is modified by an octanoyl-residue at position 3, which was expected to preclude binding of antibodies specific for this region of ghrelin. This was a particularly unlikely and surprising result, since antibodies usually recognize epitopes on proteins of the size of about 7-10 amino acids. Thus, it was expected that peptides <10 aa may not induce antibodies that efficiently recognize native ghrelin, which has an octanoyl-modification at position 3. This finding is of great therapeutic importance, since vaccination against long ghrelin-peptides (>8-12) coupled to VLPs may result in T cell responses specific for ghrelin, potentially causing autoimmune disease. Short peptides, such as peptide 1-6,1-7 or peptide 1-8 are very unlikely to be recognized by T cells and are therefore equally unlikely to induce harmful T cell responses (see ref. 39 in Bachmann and Dyer, Nature Reviews, Vol. 3, January 2004). In fact, already peptide 1-7 is too short to bind to MHC molecules and peptide 1-8 is too short to bind to MHC class II molecules, and therefore cannot induce such a T cell response. Thus, we found that peptide 1-6, peptide 1-7 and peptide 1-8 coupled to VLPs constitute safe vaccines with the surprising ability to induce potent antibody responses cross-reacting with native ghrelin. Furthermore, ghrelin-peptide coupled to VLP was capable of reducing weight gain. Furthermore, we found that surprisingly a ghrelin-peptide coupled via its C-terminus to the virus-like particles was far more potent at reducing body weight-increase than a ghrelin-peptide coupled via its N-terminus to the VLP. Thus, antibodies directed against the N-terminus are, unexpectedly, more potent than antibodies directed against the C-terminus. The present invention thus provides a prophylactic and therapeutic means for the treatment of obesity and related diseases, which is based on particular short ghrelin-derived peptides bound to a core particle, in particular on a VLP-ghrelin-peptide-conjugate and particularly on an ordered and repetitive array. These prophylactic and therapeutic compositions are able to induce high titers of anti-ghrelin antibodies in a vaccinated animal or human. Therefore, the present invention relates to ghrelin and its brain-related properties. The present invention, moreover, relates to the central effects of ghrelin in the brain, more importantly the regulation of appetite, growth hormone secretion and energy homeostasis. The antibodies induced by our vaccination strategy have been observed to be able to also bind the n-octanoylated form(s) of ghrelin. As indicated, shorter ghrelin-peptide fragments could be used, when coupled to a core particle, and alternatively administered together with adjuvant, to induce ghrelin-specific antibodies in humans and in animals. However, administration without a T-cell response-inducing adjuvant is preferred. Thus, these two different formulations (with adjuvant(+)/without adjuvant(−)) allow the choice between induction of a mixed T/B-cell-response (+) and a B-cell only (−) response).

Therefore, short peptide fragments of ghrelin, particularly the short peptides consisting of residues 1-5,1-6 (SEQ ID NO:1), 1-7 (SEQ ID NO:2) and 1-8 (SEQ ID NO:3), and in particular 1-6 (SEQ ID NO:1) and 1-8 (SEQ ID NO:3), coupled either C- or N-terminally to a core particle or a virus-like particle, respectively, and preferably coupled via their C-terminus are capable of inducing highly specific anti-ghrelin antibodies. Preferably such antibodies are capable of neutralizing peripheral circulating ghrelin before it entered the CNS and exerted an effect on growth hormone and hence, food intake.

In a preferred embodiment of the present invention, thus, the ghrelin-peptide is selected from the group of ghrelin-peptides corresponding to residues of 24-29 24-30 and 24-31, of any of the sequences set forth in SEQ ID NO:72 to 74, wherein said preferred ghrelin-peptide fragments are selected from the group consisting of (a) human ghrelin; (b) bovine ghrelin; (c) sheep ghrelin; (d) dog ghrelin; (e) cat ghrelin; (f) mouse ghrelin; (g) pig ghrelin; and (h) horse ghrelin.

More specifically, the modified VLP of the present invention was able to induce high levels of antibodies that recognize, surprisingly, the n-octanoylated form of ghrelin as shown herein, and in particular in Example 11. Furthermore, generated antibodies also recognized the alternative isoform, Ghrelin-desQ14. As a result, antibodies generated from vaccination with C- or N-terminally linked ghrelin-peptide to a core particle or, preferably to a VLP, were able to interfere with the ghrelin function in vivo, preferably by blocking the entry of n-octanoylated ghrelin into the brain and modulated food intake in mice. Therefore, the present invention focuses on vaccination strategies against ghrelin as a treatment for obesity and other related diseases.

As shown herein, and in particular in Example 12, vaccination with C- or N-terminally linked ghrelin-peptide, and in particular C-terminally linked ghrelin-peptide, to a core particle or, preferably to a VLP, leads to less body weight gain in mice, Thus, vaccines of the invention and/or antibodies induced by the vaccines of the invention, which target ghrelin and the physiological ghrelin-derived peptides, respectively, are potential therapeuticals for obesity and other related diseases.

The present invention, thus, also provides for a composition comprising: (a) a core particle with at least one first attachment site; and (b) at least one antigen or antigenic determinant with at least one second attachment site, wherein said antigen or antigenic determinant is a ghrelin-peptide of the invention, and wherein said second attachment site being selected from the group consisting of (i) an attachment site not naturally occurring with said antigen or antigenic determinant; and (ii) an attachment site naturally occurring with said antigen or antigenic determinant, wherein said second attachment site is capable of association to said first attachment site; and wherein said antigen or antigenic determinant and said core particle interact through said association, preferably to form an ordered and repetitive antigen array. Preferred embodiments of core particles suitable for use in the present invention are a virus, a virus-like particle, a bacteriophage, a virus-like particle of a RNA-phage, a bacterial pilus or flagella or any other core particle having an inherent repetitive structure, preferably such a repetitive structure which is capable of forming an ordered and repetitive antigen array in accordance with the present invention.

More specifically, the invention provides a modified VLP comprising a virus-like particle and at least one ghrelin-peptide of the invention bound thereto. Thus, in a further aspect, the invention provides a modified virus like particle (VLP) comprising: a virus like particle (VLP) and at least one peptide derived from the polypeptide ghrelin (ghrelin-peptide), wherein said ghrelin-peptide consists of a peptide with a length of 6 or 8 amino acid residues, which peptide is homologous to or identical with SEQ ID NO:1 or SEQ ID NO: 3 and wherein said VLP and said ghrelin-peptide of the invention are linked with one another. In certain preferred embodiments the linkage of the VLP and the at least one ghrelin-peptide of the invention are through at least one covalent bond, preferably through at least one non-peptide bond, and even more preferably through exclusively non-peptide bond(s).

The invention also provides a process for producing the modified VLPs of the invention. The modified VLPs and compositions of the invention are useful in the production of vaccines for the treatment of obesity and related diseases and as a pharmaceutical to prevent or cure obesity and related diseases, also to efficiently induce immune responses, in particular antibody responses. Furthermore, the modified VLPs and compositions of the invention are particularly useful to efficiently induce self-specific immune responses within the indicated context.

In the present invention, a ghrelin-peptide of the invention is bound to a core particle and VLP, respectively, preferably in an oriented manner, preferably yielding an ordered and repetitive ghrelin-peptide antigen array. Furthermore, the highly repetitive and organized structure of the core particles and VLPs, respectively, can mediate the display of the ghrelin-peptide in a highly ordered and repetitive fashion leading to a highly organized and repetitive antigen array in those preferred cases. Furthermore, binding of the ghrelin-peptide of the invention to the core particle and VLP, respectively, without being bound to any theory, may function by providing T helper cell epitopes, since the core particle and VLP is foreign to the host immunized with the core particle-ghrelin-peptide array and VLP-ghrelin-peptide array, respectively. Preferred arrays differ from prior art conjugates, in particular, in their highly organized structure, dimensions, and in the repetitiveness of the antigen on the surface of the array.

In one aspect of the invention, the ghrelin-peptide of the invention is expressed in a suitable expression host, or synthesized, while the core particle and the VLP, repespectively, is expressed and purified from an expression host suitable for the folding and assembly of the core particle and the VLP, respectively. ghrelin-peptides of the invention may be chemically synthesized. Since biologically active ghrelin contains a n-octanyolated serine at position three, chemical synthesis will be the preferred way of producing modified ghrelin-peptide for such a vaccine formulation which contains an octanoylated form of a ghrelin-peptide. The ghrelin-peptide-array of the invention is then assembled by binding the ghrelin-peptide of the invention to the core particle and the VLP, respectively.

In a further aspect, the present invention provides a composition and also a pharmaceutical composition comprising (a) the modified core particle, and in case of the pharmaceutical composition, in particular a modified VLP, also (b) an acceptable pharmaceutical carrier.

In a further aspect, the present invention provides for a pharmaceutical composition, preferably a vaccine composition, comprising (a) a virus-like particle; and (b) at least one ghrelin-peptide of the invention; and wherein said ghrelin-peptide of the invention is linked to said virus-like particle.

In still a further aspect, the present invention provides for a process for producing a modified VLP of the invention comprising (a) providing a virus-like particle; and (b) providing at least one ghrelin-peptide of the invention; (c) combining said virus-like particle and said ghrelin-peptide of the invention so that said ghrelin-peptide is bound to said virus-like particle, in particular under conditions suitable for mediating a link between the VLP and the ghrelin-peptide.

Analogously, the present invention provides a process for producing a modified core particle of the invention comprising: (a) providing a core particle with at least one first attachment site; (b) providing at least one ghrelin-peptide of the invention with at least one attachment site (further on called “second attachment site”), wherein said second attachment site being selected from the group consisting of (i) an attachment site not naturally occurring with said ghrelin-peptide of the invention; and (ii) an attachment site naturally occurring within said ghrelin-peptide of the invention; and wherein said second attachment site is capable of association to said first attachment site; and (c) combining said core particle and said at least one ghrelin-peptide of the invention, wherein said ghrelin-peptide of the invention and said core particle interact through said association, preferably to form an ordered and repetitive antigen array.

In another aspect, the present invention provides for a method of immunization comprising administering the modified VLP, the composition or pharmaceutical composition of the invention to an animal or human.

In a further aspect, the present invention provides for a use of the modified VLP, the composition or the pharmaceutical composition of the invention for the manufacture of a medicament for treatment of obesity or a related disease.

In a still further aspect, the present invention provides for a use of a modified VLP, the composition or the pharmaceutical composition of the invention for the preparation of a medicament for the therapeutic or prophylactic treatment of obesity or a related disease. Furthermore, in a still further aspect, the present invention provides for a use of a modified VLP, the composition or the pharmaceutical composition of the invention, either in isolation or in combination with other agents, for the manufacture of a composition, vaccine, drug or medicament for therapy or prophylaxis of obesity or a related disease, and/or for stimulating the mammalian immune system.

Therefore, the invention provides, in particular, vaccine compositions which are suitable for preventing and/or reducing or curing obesity or conditions related thereto. The invention further provides immunization and vaccination methods, respectively, for preventing and/or reducing or curing obesity or conditions related thereto, in animals, and in particular in pets such as cats or dogs as well as in humans. The inventive compositions may be used prophylactically or therapeutically.

In specific embodiments, the invention provides methods for preventing, curing and/or attenuating obesity or conditions related thereto which are caused or exacerbated by “self” gene products, i.e. “self antigens” as used herein. In related embodiments, the invention provides methods for inducing immunological responses in animals and individuals, respectively, which lead to the production of antibodies that prevent, cure and/or attenuate obesity or conditions related thereto, which are caused or exacerbated by “self” gene products.

As would be understood by one of ordinary skill in the art, when compositions of the invention are administered to an animal or a human, they may be in a composition which contains salts, buffers, adjuvants, or other substances which are desirable for improving the efficacy of the composition. Examples of materials suitable for use in preparing pharmaceutical compositions are provided in numerous sources including Remington's Pharmaceutical Sciences (Osol, A, ed., Mack Publishing Co. (1990)).

Compositions of the invention are said to be “pharmacologically acceptable” if their administration can be tolerated by a recipient individual. Further, the compositions of the invention will be administered in a “therapeutically effective amount” (i.e., an amount that produces a desired physiological effect).

The compositions of the present invention may be administered by various methods known in the art, but will normally be administered by injection, infusion, inhalation, oral administration or other suitable physical methods. The compositions may alternatively be administered intramuscularly, intravenously, or subcutaneously. Components of compositions for administration include sterile aqueous (e.g., physiological saline) or non-aqueous solutions and suspensions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption.

Other embodiments of the present invention will be apparent to one of ordinary skill in light of what is known in the art, the following description of the invention, and the claims.

### BRIEF DESCRIPTION OF THE DRAWINGS

FIG. 1 shows SDS-PAGE of the coupling products from the reaction of ghrelin 24-31GC or ghrelin 24-31C coupled to Qβ VLP. Lane 1 is the marker, lane 2 shows derivatized Qβ VLP, lane 3 shows Qβ-ghrelin 24-31GC in soluble fraction, lane 4 shows Qβ-ghrelin 24-31C in soluble fraction.

FIG. 2 shows the amount of I¹²⁵-ghrelin in the serum (FIG. 2A) and brain (FIG. 2B) of mice that had been previously immunized with Qβ-Ghrelin 24-31 GC or Qβ VLP as control, 30 minutes after intravenous challenge with 10 ng of I¹²⁵-ghrelin. Values are expressed as the average amount of I¹²⁵-ghrelin in the serum (ng/ml) and brain (ng/g). Brains have been corrected for the blood volume present in the brain. The error bars are SEM (standard error of the mean).

### DETAILED DESCRIPTION OF THE INVENTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are hereinafter described.

1. Definitions:

About: Unless otherwise specified, the term about refers to a value that is 10% more or less than the stated value.

Adjuvant: The term “adjuvant” as used herein refers to non-specific stimulators of the immune response or substances that allow generation of a depot in the host which when combined with the vaccine and pharmaceutical composition, respectively, of the present invention may provide for an even more enhanced immune response. A variety of adjuvants can be used. Examples include complete and incomplete Freund's adjuvant, aluminum hydroxide and modified muramyldipeptide. Further adjuvants are mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette Guerin) and Corynebacterium parvum. Such adjuvants are also well known in the art. Further adjuvants that can be administered with the compositions of the invention include, but are not limited to, Monophosphoryl lipid immunomodulator, AdjuVax 100a, QS-21, QS-18, CRL1005, Aluminum salts (Alum), MF-59, OM-174, OM-197, OM-294, and Virosomal adjuvant technology. The adjuvants can also comprise a mixture of these substances. Typically and preferably, VLP is an adjuvant. However, when the term “adjuvant” is mentioned within the context of this application, it refers to an adjuvant in addition to the VLP.

Immunologically active saponin fractions having adjuvant activity derived from the bark of the South American tree Quillaja Saponaria Molina are known in the art. For example QS21, also known as QA21, is an Hplc purified fraction from the Quillaja Saponaria Molina tree and it's method of its production is disclosed (as QA21) in U.S. Pat. No. 5,057,540. Quillaja saponin has also been disclosed as an adjuvant by Scott et al., Int. Archs. Allergy Appl. Immun., 1985, 77, 409. Monosphoryl lipid A and derivatives thereof are known in the art. A preferred derivative is 3 de-o-acylated monophosphoryl lipid A, and is known from British Patent No. 2220211. Further preferred adjuvants are described in WO00/00462, the disclosure of which is herein incorporated by reference.

However, an advantageous feature of the present invention is the high immunogenicty of the modified core particles of the invention, even in the absence of adjuvants. As already outlined herein or will become apparent as this specification proceeds, vaccines and pharmaceutical compositions devoid of adjuvants are provided, in further alternative or preferred embodiments, leading to vaccines and pharmaceutical compositions for treating obesity being devoid of adjuvants and, thus, having a superior safety profile since adjuvants may cause side-effects. The term “devoid” as used herein in the context of vaccines and pharmaceutical compositions for treating obesity refers to vaccines and pharmaceutical compositions that are used essentially without adjuvants, preferably without detectable amounts of adjuvants.

Amino acid linker: An “amino acid linker”, or also just termed “linker” within this specification, as used herein, either associates the ghrelin-peptide of the invention with the second attachment site, or more preferably, already comprises, contains or consists of the second attachment site, typically—but not necessarily—as one amino acid residue, preferably as a cysteine residue. The term “amino acid linker” as used herein, however, does not intend to imply that such an amino acid linker consists exclusively of amino acid residues, even if an amino acid linker consisting of amino acid residues is a preferred embodiment of the present invention. The amino acid residues of the amino acid linker are, preferably, composed of naturally occuring amino acids or unnatural amino acids known in the art, all-L or all-D or mixtures thereof. However, an amino acid linker comprising a molecule with a sulfhydryl group or cysteine residue is also encompassed within the invention. Such a molecule comprises preferably a C1-C6 alkyl-, cycloalkyl (C5, C6), aryl or heteroaryl moiety. However, in addition to an amino acid linker, a linker comprising preferably a C1-C6 alkyl-, cycloalkyl-(C5, C6), aryl- or heteroaryl-moiety and devoid of any amino acid(s) shall also be encompassed within the scope of the invention. Association between the ghrelin-peptide of the invention or optionally the second attachment site and the amino acid linker is preferably by way of at least one covalent bond, more preferably by way of at least one peptide bond.

Animal: As used herein, the term “animal” is meant to include, for example, humans, sheep, elks, deer, mule deer, minks, mammals, monkeys, horses, cattle, pigs, goats, dogs, cats, rats, mice, birds, chicken, reptiles, fish, insects and arachnids. Preferred animals are mammals.

Antigen: As used herein, the term “antigen” refers to a molecule capable of being bound by an antibody or a T-cell receptor (TCR) if presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens. Preferred antigens are short peptides (6-8 aa residues).

Antigenic determinant: As used herein, the term “antigenic determinant” is meant to refer to that portion of an antigen that is specifically recognized by either B- or T-lymphocytes. B-lymphocytes responding to antigenic determinants produce antibodies, whereas T-lymphocytes respond to antigenic determinants by proliferation and establishment of effector functions critical for the mediation of cellular and/or humoral immunity.

Association: As used herein, the term “association” as it applies to the first and second attachment sites, refers to the binding of the first and second attachment sites that is preferably by way of at least one non-peptide bond. The nature of the association may be covalent, ionic, hydrophobic, polar, or any combination thereof, preferably the nature of the association is covalent. The term “association” as used herein, however, shall not only encompass a direct association of the at least one first attachment site and the at least one second attachment site but also, alternatively and preferably, an indirect association of the at least one first attachment site and the at least one second attachment site through intermediate molecule(s), and hereby typically and preferably by using at least one, preferably one, heterobifunctional cross-linker.

Attachment Site, First: As used herein, the phrase “first attachment site” refers to an element of non-natural or natural origin, to which the second attachment site located on the ghrelin-peptide of the invention may associate. The first attachment site may be a protein, a polypeptide, an amino acid, a peptide, a sugar, a polynucleotide, a natural or synthetic polymer, a secondary metabolite or compound (biotin, fluorescein, retinol, digoxigenin, metal ions, phenylmethylsulfonylfluoride), or a combination thereof, or a chemically reactive group thereof. The first attachment site is located, typically and preferably on the surface, of the core particle such as, preferably the virus-like particle. Multiple first attachment sites are present on the surface of the core and virus-like particle, respectively, typically in a repetitive configuration.

Attachment Site, Second: As used herein, the phrase “second attachment site” refers to an element associated with the ghrelin-peptide of the invention to which the first attachment site located on the surface of the core particle and virus-like particle, respectively, may associate. The second attachment site of the ghrelin-peptide may be a protein, a polypeptide, a peptide, a sugar, a polynucleotide, a natural or synthetic polymer, a secondary metabolite or compound (biotin, fluorescein, retinol, digoxigenin, metal ions, phenylmethylsulfonylfluoride), or a combination thereof, or a chemically reactive group thereof. At least one second attachment site is present on the ghrelin-peptide of the invention. In certain embodiments of the invention at least one second attachment site may be added to the ghrelin-peptide of the invention. The term “ghrelin-peptide of the invention with at least one second attachment site” refers, therefore, to a ghrelin-peptide of the invention comprising at least the ghrelin-peptide of the invention and a second attachment site. However, in particular for a second attachment site, which is of non-natural origin, i.e. not naturally occurring within the ghrelin-peptide of the invention, these modified ghrelin-peptides of the invention can also comprise an “amino acid linker”.

Coat protein(s): As used herein, the term “coat protein(s)” refers to the protein(s) of a bacteriophage or a RNA-phage capable of being incorporated within the capsid assembly of the bacteriophage or the RNA-phage. However, when referring to the specific gene product of the coat protein gene of RNA-phages the term “CP” is used. For example, the specific gene product of the coat protein gene of RNA-phage Qβ is referred to as “QβCP”, whereas the “coat proteins” of bacteriophage Qβ comprise the “QβCP” as well as the A1 protein. The capsid of Bacteriophage Qβ is composed mainly of the QβCP, with a minor content of the A1 protein. Likewise, the VLP Qβcoat protein contains mainly QβCP, with a minor content of A1 protein.

Core particle: As used herein, the term “core particle” refers to a rigid structure with an inherent repetitive organization. A core particle as used herein may be the product of a synthetic process or the product of a biological process.

Coupled: The term “coupled”, as used herein, refers to attachment by covalent bonds or by strong non-covalent interactions, typically and preferably to attachment by covalent bonds. Any method normally used by those skilled in the art for the coupling of biologically active materials can be used in the present invention.

Effective Amount: As used herein, the term “effective amount” refers to an amount necessary or sufficient to realize a desired biologic effect. An effective amount of the composition would be the amount that achieves this selected result, and such an amount could be determined as a matter of routine by a person skilled in the art. For example, an effective amount for treating an immune system deficiency could be that amount necessary to cause activation of the immune system, resulting in the development of an antigen specific immune response upon exposure to antigen. The term is also synonymous with “sufficient amount.”

The effective amount for any particular application can vary depending on such factors as the disease or condition being treated, the particular composition being administered, the size of the subject, and/or the severity of the disease or condition. One of ordinary skill in the art can empirically determine the effective amount of a particular composition of the present invention without necessitating undue experimentation.

Epitope: As used herein, the term “epitope” refers to continuous or discontinuous portions of a polypeptide having antigenic or immunogenic activity in an animal, preferably a mammal, and most preferably in a human. An epitope is recognized by an antibody or a T cell through its T cell receptor in the context of an MHC molecule. An “immunogenic epitope,” as used herein, is defined as a portion of a polypeptide that elicits an antibody response or induces a T-cell response in an animal, as determined by any method known in the art. (See, for example, Geysen et al., Proc. Natl. Acad. Sci. USA 81:3998-4002 (1983)). The term “antigenic epitope,” as used herein, is defined as a portion of a protein to which an antibody can immunospecifically bind its antigen as determined by any method well known in the art. Immunospecific binding excludes non-specific binding but does not necessarily exclude cross-reactivity with other antigens. Antigenic epitopes need not necessarily be immunogenic. Antigenic epitopes can also be T-cell epitopes, in which case they can be bound immunospecifically by a T-cell receptor within the context of an MHC molecule.

An epitope typically comprise 7-10 amino acids in a spatial conformation which is unique to the epitope. If the epitope is an organic molecule, it may be as small as Nitrophenyl. Preferred epitopes are the ghrelin-peptides of the invention, which are believed to be B-cell epitopes.

Fusion: As used herein, the term “fusion” refers to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini.

Ghrelin: The term “ghrelin” as used herein refers to a protein encoded by a ghrelin gene. As used herein ghrelin includes all forms of ghrelins known in humans, cats, dogs and all domesticated animals as well as of other animals. Ghrelin, as used herein, includes ghrelin with or without a n-octanoyl-modification. Moreover, ghrelin also includes all splice variants that exist of ghrelin. In addition, due to high sequence homology between ghrelins of different species (only 2 aa exchanged between rat and human ghrelin (Kojima et al., Nature 402:656-660 (1999)), all natural variants of ghrelin with more than 80% identity, preferably more than 90%, more preferably more than 95%, and even more preferably more than 99% with human ghrelin are referred to as “ghrelin” herein.

As used herein, the term “ghrelin-peptide” or “ghrelin peptide of the invention” is defined as a peptide which has a length of 6-8 amino acid residues, which peptide is homologous to, or identical with, SEQ ID NO:1 (GSSFLS), SEQ ID NO:2 (GSSFLSP) or SEQ ID NO:3 (GSSFLSPE). A homologous peptide is such a peptide which (i) is derived from a ghrelin of another animal, particularly a mammalian ghrelin, like e.g. feline or canine ghrelin, and represents those amino acid residues that correspond to SEQ ID NO:1, SEQ ID NO:2 or SEQ ID NO:3; or (ii) differs from SEQ ID NO:1, SEQ ID NO:2 or SEQ ID NO:3 at only two, preferably only one, position from SEQ ID NO:1, SEQ ID NO:2 or SEQ ID NO:3, which difference is a difference in amino acid nature at a particular position such as a substitution of an amino acid or a modification of an amino acid such as an acylation or a glycosylation, and hereby preferably a substitution, and hereby even more preferably a conservative substitution, and which difference is, preferably, not a difference in length. The homologous peptides, and hereby in particular the homologous peptides in accordance with (i), are identifiable to a skilled person by way of aligning human ghrelin with said ghrelin of the other animal. The term “ghrelin-peptide” or “ghrelin peptide of the invention”, as used herein, preferably refers a peptide which has a length of 6 or 8 amino acid residues, which peptide is homologous to, or identical with, SEQ ID NO:1 (GSSFLS) or SEQ ID NO:3 (GSSFLSPE). A homologous peptide in accordance with preferred embodiments of the present invention is such a peptide which (i) is derived from a ghrelin of another animal, particularly a mammalian ghrelin, like e.g. feline or canine ghrelin, and represents those amino acid residues that correspond to SEQ ID NO:1 or SEQ ID NO:3. In such cases, where the ghrelin-peptides of the invention are comprised within a larger context, i.e. a fusion polypeptide or a ghrelin-peptide with an added linker peptide or attachment site, the ghrelin-peptide, for example, of SEQ ID NO. 1 is, preferably, not followed by a proline residue and the ghrelin-peptide, for example, of SEQ ID NO. 3 is, preferably, not followed by a histidine residue. The ghrelin-peptide may be obtained by recombinant expression in eukaryotic or prokaryotic expression systems as ghrelin-peptide alone, but preferably as a fusion with other amino acids or proteins, e.g. to facilitate folding, expression or solubility of the ghrelin-peptide or to facilitate purification of the ghrelin-peptide. Preferred are fusions between ghrelin-peptides and subunit proteins of VLPs or capsids. In such a case, one or more amino acids may be added N- or C-terminally to ghrelin-peptides, but it is preferred that the ghrelin-peptide is at the N-terminus of a fusion polypeptide, i.e. coupled or linked via its own C-terminus to its fusion partner.

Very preferably, to enable coupling of ghrelin-peptides to subunit proteins of VLPs or capsids or core particles, at least one second attachment site may be added to the ghrelin-peptide. Alternatively ghrelin-peptides may be synthesized using methods known to the art, in particular by organic-chemical peptide synthesis. Such peptides may even contain amino acids which are not present in the corresponding ghrelin protein. The peptides may be modified by n-octanoylation, but this modification is surprisingly not necessary for induction of effective antibodies by the modified VLP, compositions or vaccines of the present invention.

Residue: As used herein, the term “residue” is meant to mean a specific amino acid in a polypeptide backbone or side chain.

Immune response: As used herein, the term “immune response” refers to a humoral immune response and/or cellular immune response leading to the activation or proliferation of B- and/or T-lymphocytes and/or and antigen presenting cells. In some instances, however, the immune responses may be of low intensity and become detectable only when using at least one substance in accordance with the invention. “Immunogenic” refers to an agent used to stimulate the immune system of a living organism, so that one or more functions of the immune system are increased and directed towards the immunogenic agent. A substance which “enhances” an immune response refers to a substance in which an immune response is observed that is greater or intensified or deviated in any way with the addition of the substance when compared to the same immune response measured without the addition of the substance. For example, the lytic activity of cytotoxic T cells can be measured, e.g. using a 51Cr release assay in samples obtained with and without the use of the substance during immunization, as disclosed in Bachmann et al., (1997) “LCMV-specific CTL responses”, in Immunology Methods Manual, Academic Press Ltd. The amount of the substance at which the CTL lytic activity is enhanced as compared to the CTL lytic activity without the substance is said to be an amount sufficient to enhance the immune response of the animal to the antigen. In a preferred embodiment, the immune response in enhanced by a factor of at least about 2, more preferably by a factor of about 3 or more. The amount or type of cytokines secreted may also be altered. Alternatively, the amount of antibodies induced or their subclasses may be altered.

Immunization: As used herein, the terms “immunize” or “immunization” or related terms refer to conferring the ability to mount a substantial immune response (comprising antibodies and/or cellular immunity such as effector CTL) against a target antigen or epitope. These terms do not require that complete immunity be created, but rather that an immune response be produced which is substantially greater than baseline. For example, a mammal may be considered to be immunized against a target antigen if the cellular and/or humoral immune response to the target antigen occurs following the application of methods of the invention.

Iinked: As used herein, the term “linked” as well as the term “bound”, which is herein used equivalently, refers to binding or attachment that may be covalent, e.g., by chemically coupling, or non-covalent, e.g., ionic interactions, hydrophobic interactions, hydrogen bonds, etc. Covalent bonds can be, for example, ester, ether, phosphoester, amide, peptide, imide, carbon-sulfur bonds, carbon-phosphorus bonds, and the like. The term “linked” as used herein, and in particular if referring to the linkage between the virus-like particle and the at least one ghrelin-peptide shall not only encompass a direct linkage of the VLP and the ghrelin-peptide of the invention, but also, alternatively and preferably, an indirect linkage of the VLP and the ghrelin-peptide of the invention through intermediate molecule(s), and hereby typically and preferably by using at least one, preferably one, heterobifunctional cross-linker.

Natural origin: As used herein, the term “natural origin” means that the whole or parts thereof are not synthetic and exist or are produced in nature.

Non-natural: As used herein, the term generally means not from nature, more specifically, the term means from the hand of man.

Non-natural origin: As used herein, the term “non-natural origin” generally means synthetic or not from nature; more specifically, the term means from the hand of man.

Ordered and repetitive antigen or antigenic determinant array: As used herein, the term “ordered and repetitive antigen or antigenic determinant array” generally refers to a repeating pattern of antigen or antigenic determinant, characterized by a typically and preferably uniform spacial arrangement of the antigens or antigenic determinants with respect to the core particle and virus-like particle, respectively. In one embodiment of the invention, the repeating pattern may be a geometric pattern. Typical and preferred examples of suitable ordered and repetitive antigen or antigenic determinant arrays are those which possess strictly repetitive paracrystalline orders of antigens or antigenic determinants, preferably with spacings of 1 to 30 nanometers, preferably 2 to 15 nanometers, even more preferably 2 to 10 nanometers, even again more preferably 2 to 8 nanometers, and further again more preferably 2 to 7 nanometers.

Pili: As used herein, the term “pili” (singular being “pilus”) refers to extracellular structures of bacterial cells composed of protein monomers (e.g., pilin monomers) which are organized into ordered and repetitive patterns. Further, pili are structures which are involved in processes such as the attachment of bacterial cells to host cell surface receptors, inter-cellular genetic exchanges, and cell-cell recognition. Examples of pili include Type-1 pili, P-pili, F1C pili, S-pili, and 987P-pili. Additional examples of pili are set out below.

Pilus-like structure: As used herein, the phrase “pilus-like structure” refers to structures having characteristics similar to that of pili and composed of protein monomers. One example of a “pilus-like structure” is a structure formed by a bacterial cell which expresses modified pilin proteins that do not form ordered and repetitive arrays that are identical to those of natural pili.

Polypeptide: As used herein, the term “polypeptide” refers to a molecule composed of monomers (amino acids) linearly linked by amide bonds (also known as peptide bonds). It indicates a molecular chain of amino acids and does not refer to a specific length of the product. Thus, peptides, dipeptides, tripeptides, oligopeptides and proteins are included within the definition of polypeptide. Preferred peptides of the invention are pentapeptides, hexapeptides, heptapeptides and octapeptides. A polypeptide is composed of more amino acid residues than an octapeptide, for the purposes of this invention. This term is also intended to refer to post-expression modifications of the polypeptide or peptide, for example, glycosylations, acetylations, phosphorylations, and the like. A recombinant or derived polypeptide or peptide is not necessarily translated from a designated nucleic acid sequence. It may also be generated in any manner, including chemical synthesis, which is preferred for peptides.

Self antigen: As used herein, the tem “self antigen” refers to proteins encoded by the host's DNA and products generated by proteins or RNA encoded by the host's DNA are defined as self. In addition, proteins that result from a combination of two or several self-molecules or that represent a fraction of a self-molecule and proteins that have a high homology two self-molecules as defined above (>95%, preferably >97%, more preferably >99%) may also be considered self.

Treatment: As used herein, the terms “treatment”, “treat”, “treated” or “treating” refer to prophylaxis and/or therapy. When used with respect to an infectious disease, for example, the term refers to a prophylactic treatment which increases the resistance of a subject to infection with a pathogen or, in other words, decreases the likelihood that the subject will become infected with the pathogen or will show signs of illness attributable to the infection, as well as a treatment after the subject has become infected in order to fight the infection, e.g., reduce or eliminate the infection or prevent it from becoming worse. When used with respect to obesity or related diseases, the term “treatment” refers to a prophylactic or therapeutic treatment which increases the resistance of a subject against, and/or which reverts obesity.

Vaccine: As used herein, the term “vaccine” refers to a formulation which contains the modified core particle, and in particular the modified VLP of the present invention and which is in a form that is capable of being administered to an animal. Typically, the vaccine comprises a conventional saline or buffered aqueous solution medium in which the composition of the present invention is suspended or dissolved. In this form, the composition of the present invention can be used conveniently to prevent, ameliorate, or otherwise treat a condition. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses. Typically, the modified core particle of the invention, and preferably, the modified VLP of the invention, preferably induces a predominant B-cell response, more preferably a B-cell response only, which is a further advantage.

Optionally, the vaccine of the present invention additionally includes an adjuvant which can be present in either a minor or major proportion relative to the compound of the present invention.

Virus-like particle (VLP): As used herein, the term “virus-like particle” refers to a structure resembling a virus particle. Moreover, a virus-like particle in accordance with the invention is non-replicative and noninfectious since it lacks all or part of the viral genome function, in particular the replicative and infectious components of the viral genome. The viral genome function can be inactivated by physical or chemical methods, such as UV irradiation or formaldehyde treatment, or preferably by genetic engineering method to delete or mutate the genes responsible for infection and/or replication. A virus-like particle in accordance with the invention may contain nucleic acid distinct from their genome. A typical and preferred embodiment of a virus-like particle in accordance with the present invention is a viral capsid such as the viral capsid of the corresponding virus, bacteriophage, or RNA-phage. The terms “viral capsid” or “capsid”, as interchangeably used herein, refer to a macromolecular assembly composed of viral protein subunits. Typically and preferably, the viral protein subunits assemble into a viral capsid and capsid, respectively, having a structure with an inherent repetitive organization, wherein said structure is, typically, spherical or tubular. For example, the capsids of RNA-phages or HBcAgs have a spherical form of icosahedral symmetry. The term “capsid-like structure” as used herein, refers to a macromolecular assembly composed of viral protein subunits resembling the capsid morphology in the above defined sense but deviating from the typical symmetrical assembly while maintaining a sufficient degree of order and repetitiveness.

Virus-like particle of a bacteriophage: As used herein, the term “virus-like particle of a bacteriophage” as well as the term “virus-like particle derived from a bacteriophage”, which is herein used equivalently, refers to a virus-like particle resembling the structure of a bacteriophage, being non replicative and noninfectious, and lacking at least the gene or genes encoding for the replication machinery of the bacteriophage, and typically also lacking the gene or genes encoding the protein or proteins responsible for viral attachment to or entry into the host. This definition should, however, also encompass virus-like particles of bacteriophages, in which the aforementioned gene or genes are still present but inactive, and, therefore, also leading to non-replicative and noninfectious virus-like particles of a bacteriophage. Most VLPs derived from RNA-phages exhibit icosahedral symmetry and consist of 180 subunits. Within this present disclosure the term “subunit” and “monomer” are interexchangeably and equivalently used within this context.

VLP of RNA phage coat protein: The capsid structure formed from the self-assembly of 180 subunits of RNA phage coat protein and optionally containing host RNA is referred to as a “VLP of RNA phage coat protein.” A specific example is the VLP of Qβ coat protein. In this particular case, the VLP of Qβcoat protein may either be assembled exclusively from QβCP subunits (generated by expression of a QβCP gene containing, for example, a TAA stop codon precluding any expression of the longer A1 protein through suppression, see Kozlovska, T. M., et al., Intervirology 39: 9-15 (1996)), or additionally contain A1 protein subunits in the capsid assembly.

Virus particle: The term “virus particle” as used herein refers to the morphological form of a virus. In some virus types it comprises a genome surrounded by a protein capsid; others have additional structures (e.g., envelopes, tails, etc.).

One, a, or an: When the terms “one,” “a,” or “an” are used in this disclosure, they mean “at least one” or “one or more,” unless otherwise indicated.

As will be clear to those skilled in the art, certain embodiments of the invention involve the use of recombinant nucleic acid technologies such as cloning, polymerase chain reaction, the purification of DNA and RNA, the expression of recombinant proteins in prokaryotic and eukaryotic cells, etc. Such methodologies are well known to those skilled in the art and can be conveniently found in published laboratory methods manuals (e.g., Sambrook, J. et al., eds., Molecular Cloning, A Laboratory Manual, 2nd edition, Cold Spring Harbor Laboratory Press, Cold Spring Harbor, N.Y. (1989); Ausubel, F. et al., eds., Current Protocols in Molecular Biology, John H. Wiley & Sons, Inc. (1997)). Fundamental laboratory techniques for working with tissue culture cell lines (Celis, J., ed., Cell Biology, Academic Press, 2nd edition, (1998)) and antibody-based technologies (Harlow, E. and Lane, D., Antibodies: A Laboratory Manual, Cold Spring Harbor Laboratory, Cold Spring Harbor, N.Y. (1988); Deutscher, M. P., “Guide to Protein Purification,” Meth. Enzymol. 128, Academic Press San Diego (1990); Scopes, R. K., Protein Purification Principles and Practice, 3rd ed., Springer-Verlag, New York (1994)) are also adequately described in the literature, all of which are incorporated herein by reference.

2. Compositions and Methods for Enhancing an Immune Response

The disclosed invention provides compositions and methods for enhancing an immune response against ghrelin or a ghrelin-peptide in an animal or in a human. Compositions of the invention comprise, or alternatively consist of (a) a core particle, and preferably a VLP; and (b) at least one ghreling-peptide, wherein said ghrelin-peptide consists of a peptide with a length of 6 or 8 amino acid residues which peptide is homologous to or identical with SEQ ID NOs: 1 (GSSFLS) or 3 (GSSFLSPE) and wherein a) and b) are linked with one another. More specifically, the modified core particle, and preferably the modified VLP of the invention comprise, or alternatively consist of, a virus-like particle and at least one ghrelin-peptide of the invention. Preferably, the ghrelin-peptide of the invention is bound to the virus-like particle so as to form an ordered and repetitive antigen-VLP-array. Furthermore, the invention conveniently enables the practitioner to construct such a composition, inter alia, for treatment and/or prophylactic prevention of obesity.

In one embodiment, the core particle comprises, or is selected from a group consisting of, a virus, a bacterial pilus, a structure formed from bacterial pilin, a bacteriophage, a virus-like particle, a virus-like particle of a RNA phage, a viral capsid particle or a recombinant form thereof. Preferably said core particle, and even more preferably said VLP comprises, or is a recombinant virus-like particle.

Any virus known in the art having an ordered and repetitive coat and/or core protein structure may be selected as a core particle of the invention; examples of suitable viruses include sindbis and other alphaviruses, rhabdoviruses (e.g. vesicular stomatitis virus), picornaviruses (e.g., human rhino virus, Aichi virus), togaviruses (e.g., rubella virus), orthomyxoviruses (e.g., Thogoto virus, Batken virus, fowl plague virus), polyomaviruses (e.g., polyomavirus BK, polyomavirus JC, avian polyomavirus BFDV), parvoviruses, rotaviruses, Norwalk virus, foot and mouth disease virus, a retrovirus, Hepatitis B virus, Tobacco mosaic virus, Flock House Virus, and human Papilomavirus, and preferably a RNA phage, wherein preferably said RNA phage is selected from a group consisting of: bacteriophage Qβ, bacteriophage R17, bacteriophage M11, bacteriophage MX1, bacteriophage NL95, bacteriophage fr, bacteriophage GA, bacteriophage SP, bacteriophage MS2, bacteriophage f2, bacteriophage PP7 and AP205. (for example, see Table 1 in Bachmann, M. F. and Zinkernagel, R. M., Immunol. Today 17:553-558 (1996)).

In a further embodiment, the invention utilizes genetic engineering of a virus to create a fusion between an ordered and repetitive viral coat protein and a ghrelin-peptide of the invention, or alternatively a first attachment site being comprised by, or alternatively or preferably being a heterologous protein, peptide, antigenic determinant or a reactive amino acid residue of choice, and a ghrelin-peptide of the invention with an added second attachment site. Other genetic manipulations known to those in the art may be included in the construction of the inventive compositions; for example, it may be desirable to restrict the replication ability of the recombinant virus through genetic mutation. Furthermore, the virus used for the present invention is replication incompetent due to chemical or physical inactivation or, as indicated, due to lack of a replication competent genome and/or genome function. The viral protein selected for fusion to the first attachment site should have an organized and repetitive structure. Such an organized and repetitive structure includes paracrystalline organizations with a spacing of 5-30 nm, preferably 5-15 nm, on the surface of the virus. The creation of this type of fusion protein will result in multiple, ordered and repetitive ghrelin-peptide of the invention, or alternatively first attachment sites on the surface of the virus and reflect the normal organization of the native viral protein. As will be understood by those in the art, the first attachment site may be or be a part of any suitable protein, polypeptide, sugar, polynucleotide, peptide (amino acid), natural or synthetic polymer, a secondary metabolite or combination thereof that may serve to specifically attach the at least one ghrelin-peptide of the invention leading, preferably, to an ordered and repetitive antigen array. Of course, direct fusions between the viral coat protein on the ghrelin-peptide of the invention can be made without the introduction of first and/or second attachment sites, in which case the first attachment site is the natural amino acid of the viral coat protein, and the second attachment site is the natural amino acid of the ghrelin-peptide of the invention or the natural amino acid of the amino acid linker bound, preferably fused to, the ghrelin-peptid, and the first and the second attachment site are linked by a peptide bond.

In another embodiment of the invention, the core particle is a recombinant alphavirus, and more specifically, a recombinant Sinbis virus. Several members of the alphavirus family, Sindbis (Xiong, C. et al., Science 243:1188-1191 (1989); Schlesinger, S., Trends Biotechnol. 11:18-22 (1993)), Semliki Forest Virus (SFV) (Liljeström, P. & Garoff, H., Bio/Technology 9:1356-1361 (1991)) and others (Davis, N. L. et al., Virology 171:189-204 (1989)), have received considerable attention for use as virus-based expression vectors for a variety of different proteins (Lundstrom, K., Curr. Opin. Biotechnol. 8:578-582 (1997); Liljeström, P., Curr. Opin. Biotechnol. 5:495-500 (1994)) and as candidates for vaccine development. Recently, a number of patents have issued directed to the use of alphaviruses for the expression of heterologous proteins and the development of vaccines (see U.S. Pat. Nos. 5,766,602; 5,792,462; 5,739,026; 5,789,245 and 5,814,482). The construction of the modified alphaviral core particles of the invention may be done by means generally known in the art of recombinant DNA technology, as described by the aforementioned articles, which are incorporated herein by reference.

Any virus known in the art having an ordered and repetitive structure may be selected as a VLP of the invention. Illustrative DNA or RNA viruses, the coat or capsid protein of which can be used for the preparation of VLPs have been disclosed in WO 2004/009124 on page 25, line 10-21, on page 26, line 11-28, and on page 28, line 4 to page 31, line 4. These disclosures are incorporated herein by way of reference.

In other embodiments, a bacterial pilin, a subportion of a bacterial pilin, or a fusion protein which contains either a bacterial pilin or subportion thereof is used to prepare modified core particles and compositions and vaccine compositions, respectively, of the invention. Examples of pilin proteins include pilins produced by Escherichia coli, Haemophilus influenzae, Neisseria meningitidis, Neisseria gonorrhoeae, Caulobacter crescentus, Pseudomonas stutzeri, and Pseudomonas aeruginosa. The amino acid sequences of pilin proteins suitable for use with the present invention include those set out in GenBank reports AJ000636, AJ132364, AF229646, AF051814, AF051815), and X00981, the entire disclosures of which are incorporated herein by reference.

Specific and preferred examples of pilin proteins suitable for use in the present invention are disclosed in WO 02/056905 on page 41, line 13 to line 21 and on page 41 line 25 to page 43, line 22 and herein are incorporated by way of reference.

In most instances, the pili or pilus-like structures used in compositions and vaccine compositions, respectively, of the invention will be composed of single type of a pilin subunit. However, the compositions of the invention also include compositions and vaccines comprising pili or pilus-like structures formed from heterogenous pilin subunits. Possible methods of expression of those preferred embodiments of the invention are are disclosed in WO 02/056905 on page 43 line 28 to page 44, line 6.

In addition, the ghrelin-peptide of the invention can be linked to bacterial pili or pilus-like structures by at least one covalent bond. In one preferred embodiment, said at least covalent bond is a non-peptide bond. In a further preferred embodiment, the first attachment site is a lysine naturally occurring within or engineered to pillin. In another further preferred embodiment, the second attachment site is a cysteine added to the ghrelin peptide.

In another preferred embodiment, said at least one covalent bond is a peptide bond. Bacterial cells which produce pili or pilus-like structures used in the compositions of the invention can be genetically engineered to generate pilin proteins which are fused to a ghrelin-peptide of the invention. Such fusion proteins which form pili or pilus-like structures are suitable for use in vaccine compositions of the invention.

In a preferred embodiment, the core particle is a virus-like particle, preferably wherein the virus-like particle is a recombinant virus-like particle. The skilled artisan can produce VLPs using recombinant DNA technology and virus coding sequences which are readily available to the public.

Examples of VLPs include, but are not limited to, the capsid proteins of Hepatitis B virus (Ulrich, et al., Virus Res. 50:141-182 (1998)), measles virus (Warnes, et al., Gene 160:173-178 (1995)), Sindbis virus, rotavirus (U.S. Pat. No. 5,071,651 and U.S. Pat. No. 5,374,426), foot-and-mouth-disease virus (Twomey, et al., Vaccine 13:1603 1610, (1995)), Norwalk virus (Jiang, X., et al., Science 250:1580 1583 (1990); Matsui, S. M., et al., J. Clin. Invest. 87:1456 1461 (1991)), the retroviral GAG protein (WO 96/30523), the retrotransposon Ty protein p1, the surface protein of Hepatitis B virus (WO 92/11291), human papilloma virus (WO 98/15631), RNA phages, Ty, fr-phage, GA-phage, AP205-phage and Qβ-phage.

As will be readily apparent to those skilled in the art, the VLP of the invention is not limited to any specific form. The particle can be synthesized chemically or through a biological process, which can be natural or non-natural. By way of example, this type of embodiment includes a virus-like particle or a recombinant form thereof.

In a more specific embodiment, the VLP can comprise, or alternatively essentially consist of, or alternatively consist of recombinant polypeptides, or fragments thereof being capable of assembling into a VLP, being selected from recombinant polypeptides of Rotavirus, recombinant polypeptides of Norwalk virus, recombinant polypeptides of Alphavirus, recombinant polypeptides of Foot and Mouth Disease virus, recombinant polypeptides of measles virus, recombinant polypeptides of Sindbis virus, recombinant polypeptides of Polyoma virus, recombinant polypeptides of Retrovirus, recombinant polypeptides of Hepatitis B virus (e.g., a HBcAg), recombinant polypeptides of Tobacco mosaic virus, recombinant polypeptides of Flock House Virus, recombinant polypeptides of human Papillomavirus, recombinant polypeptides of bacteriophages, recombinant polypeptides of RNA phages, recombinant polypeptides of Ty, recombinant polypeptides of fr-phage, recombinant polypeptides of GA-phage and recombinant polypeptides of Qβ-phage. The virus-like particle can further comprise, or alternatively essentially consist of, or alternatively consist of, one or more fragments of such polypeptides, as well as mutants of such polypeptides, on the condition that said fragment of such polypeptide or said mutant of such polypeptide are capable of assembling into a VLP. Variants of polypeptides can share, for example, at least 80%, 85%, 90%, 95%, 97%, or 99% identity at the amino acid level with their wild-type counterparts.

In one preferred embodiment, the virus-like particle of the invention is of Hepatitis B virus. The preparation of Hepatitis B virus-like particles have been disclosed, inter alia, in WO 00/32227, WO 01/85208 and in WO 01/056905. All three documents are explicitly incorporated herein by way of reference. Other variants of HBcAg suitable for use in the practice of the present invention have been disclosed in page 34-39 WO 01/056905.

In one further preferred embodiments of the invention, a lysine residue is introduced into the HBcAg polypeptide, to mediate the linking of ghrelin-ppetide of the invention to the VLP of HBcAg. In preferred embodiments, VLPs and compositions of the invention are prepared using a HBcAg comprising, or alternatively consisting of, amino acids 1-144, or 1-149, 1-185 of SEQ ID NO:25, which is modified so that the amino acids at positions 79 and 80 are replaced with a peptide having the amino acid sequence of Gly-Gly-Lys-Gly-Gly. This modification changes the SEQ ID NO:25 to SEQ ID NO:26. In further preferred embodiments, the cysteine residues at positions 48 and 110 of SEQ ID NO:25, or its corresponding fragments, preferably 1-144 or 1-149, are mutated to serine. The invention further includes compositions comprising Hepatitis B core protein mutants having above noted corresponding amino acid alterations. The invention further includes compositions and vaccines, respectively, comprising HBcAg polypeptides which comprise, or alternatively consist of, amino acid sequences which are at least 80%, 85%, 90%, 95%, 97% or 99% identical to SEQ ID NO:26.

In a preferred embodiment, the VLP is a virus-like particle of an RNA bacteriophage. In a further preferred embodiment, the virus-like particle comprises, preferably consists essentially of, or alternatively consists of recombinant proteins, or fragments thereof, of a RNA-phage. In a further preferred embodiment, the virus-like particle comprises, preferably consists essentially of, or alternatively consists of recombinant coat proteins, or fragments thereof, of a RNA-phage. Preferably, the RNA-phage is selected from the group consisting of a) bacteriophage Qβ; b) bacteriophage R17; c) bacteriophage fr; d) bacteriophage GA; e) bacteriophage SP; f) bacteriophage MS2; g) bacteriophage M11; h) bacteriophage MX1; i) bacteriophage NL95; k) bacteriophage f2; 1) bacteriophage PP7, and m) bacteriophage AP205.

In another preferred embodiment of the present invention, the virus-like particle comprises, or alternatively consists essentially of, or alternatively consists of recombinant proteins, or fragments thereof being capable of assembling into a VLP, of the RNA-bacteriophage Qβ or of the RNA-bacteriophage fr, or of the RNA-bacteriophage AP205, preferably of RNA-bacteriophage Qβ.

In one preferred embodiment, the VLP comprises, or alternatively consists of, recombinant proteins, or fragments thereof, of a RNA-phage, and wherein preferably said recombinant proteins comprise, or alternatively consist essentially of, or alternatively consist of coat proteins of RNA phages.

In another preferred embodiment of the present invention, the virus-like particle comprises, or alternatively consists of, recombinant proteins, preferably recombinant coat protein or fragments thereof being capable of assembling into a VLP, of RNA-phage Qβ, fr, AP205 or GA.

RNA-phage coat proteins forming capsids or VLPs, or fragments of the bacteriophage coat proteins compatible with self-assembly into a capsid or a VLP, are, therefore, further preferred embodiments of the present invention. Bacteriophage Qβ coat proteins, for example, can be expressed recombinantly in E. coli. Specific preferred examples of bacteriophage coat proteins which can be used to prepare compositions of the invention include the coat proteins of RNA bacteriophages such as bacteriophage Qβ (SEQ ID NO:4 and SEQ ID NO:5), bacteriophage R17 (SEQ ID NO:6), bacteriophage fr (SEQ ID NO:7), bacteriophage GA (SEQ ID NO:8), bacteriophage SP (SEQ ID NO:9 and SEQ ID NO:10), bacteriophage MS2 (SEQ ID NO:11), bacteriophage M11 (SEQ ID NO:12), bacteriophage MX1 (SEQ ID NO:13), bacteriophage NL95 (SEQ ID NO:14), bacteriophage f2 (SEQ ID NO:15), bacteriophage PP7 (SEQ ID NO:16), and bacteriophage AP205 (SEQ ID NO:28). Furthermore, the A1 protein of bacteriophage Qβ (SEQ ID NO:5) or C-terminal truncated forms missing as much as 100, 150 or 180 amino acids from its C-terminus of A1 may be incorporated in a capsid assembly of Qβ coat proteins. Generally, the percentage of Qβ A1 protein relative to Qβ CP in the capsid assembly will be limited, in order to ensure capsid formation. In a further prefererd embodiment of the present invention, the coat proteins of RNA phages having an amino acid sequence selected from the group consisting of SEQ ID NO:4; a mixture of SEQ ID NO:4 and SEQ ID NO:5; SEQ ID NO:6; SEQ ID NO:7; SEQ ID NO:8; SEQ ID NO:9; a mixture of SEQ ID NO:9 and SEQ ID NO:10; SEQ ID NO:1; SEQ ID NO:12; SEQ ID NO:13; SEQ ID NO:14; SEQ ID NO:15; SEQ ID NO:16; and SEQ ID NO:28.

Qβ coat protein has also been found to self-assemble into capsids when expressed in E. coli (Kozlovska T M. et al., GENE 137:133-137 (1993)). The capsid contains 180 copies of the coat protein, which are linked in covalent pentamers and hexamers by disulfide bridges (Golmohammadi, R. et al., Structure 4:543-5554 (1996)) leading to a remarkable stability of the capsid of Qβ coat protein. Capsids or VLPs made from recombinant Qβ coat protein may contain, however, subunits not linked via disulfide links to other subunits within the capsid, or incompletely linked. However, typically more than about 80% of the subunits are linked via disulfide bridges to each other within the VLP. Qβ capsid protein also shows unusual resistance to organic solvents and denaturing agents. Surprisingly, we have observed that DMSO and acetonitrile concentrations as high as 30%, and Guanidinium concentrations as high as 1 M do not affect the stability of the capsid. The high stability of the capsid of Qβ coat protein is an advantageous feature, in particular, for its use in immunization and vaccination of mammals and humans in accordance of the present invention.

Further preferred virus-like particles of RNA-phages, in particular of Qβ in accordance of this invention are disclosed in WO 02/056905, the disclosure of which is herewith incorporated by reference in its entirety.

Further RNA phage coat proteins have also been shown to self-assemble upon expression in a bacterial host (Kastelein, R A. et al., Gene 23:245-254 (1983), Kozlovskaya, T M. et al., Dokl. Akad. Nauk SSSR 287:452-455 (1986), Adhin, MR. et al., Virology 170:238-242 (1989), Ni, CZ., et al., Protein Sci. 5:2485-2493 (1996), Priano, C. et al., J. Mol. Biol. 249:283-297 (1995)). The Qβ phage capsid contains, in addition to the coat protein, the so called read-through protein A1 and the maturation protein A2. A1 is generated by suppression at the UGA stop codon and has a length of 329 aa. The capsid of phage Qβ recombinant coat protein used in the invention is devoid of the A2 lysis protein, and contains RNA from the host.

In a further preferred embodiment of the present invention, the virus-like particle comprises, or alternatively consists essentially of, or alternatively consists of recombinant proteins, or fragments thereof, of a RNA-phage, wherein the recombinant proteins comprise, alternatively consist essentially of or alternatively consist of mutant coat proteins of a RNA phage, preferably of mutant coat proteins of the RNA phages mentioned above. In one embodiment, the mutant coat proteins are fusion proteins with a ghrelin-peptide of the invention. In another preferred embodiment, the mutant coat proteins of the RNA phage have been modified by removal of at least one, or alternatively at least two, lysine residue by way of substitution, or by addition of at least one lysine residue by way of substitution; alternatively, the mutant coat proteins of the RNA phage have been modified by deletion of at least one, or alternatively at least two, lysine residue, or by addition of at least one lysine residue by way of insertion. The deletion, substitution or addition of at least one lysine residue allows varying the degree of coupling, i.e. the amount of ghrelin peptides per subunits of the VLP of the RNA-phages, in particular, to match and tailor the requirements of the vaccine. Thus, in a further preferred embodiment of the present invention, the virus-like particle of an RNA bacteriophage comprises one or more coat proteins of said RNA phage modified by deletion or substitution to remove at least one naturally occurring lysine residue, or that have been modified by insertion or substitution to add at least one lysine resid.

In a preferred embodiment of the present invention, on average at least 1.0 ghrelin peptide per subunit are linked to the VLP of the RNA-phage. This value is calculated as an average over all the subunits or monomers of the VLP of the RNA-phage. In a further preferred embodiment of the present invention, at least 1.1, 1.2, 1.3, 1.4, 1.5, 1.6, 1.7, 1.8, 1.9 or at least 2.0 ghrelin peptides are linked to the VLP of the RNA-phages as being calculated as an coupling average over all the subunits or monomers of the VLP of the RNA-phage.

In another preferred embodiment, the virus-like particle comprises, or alternatively consists essentially of, or alternatively consists of recombinant proteins, or fragments thereof, of the RNA-bacteriophage Qβ, wherein the recombinant proteins comprise, or alternatively consist essentially of, or alternatively consist of coat proteins having an amino acid sequence of SEQ ID NO:4, or a mixture of coat proteins having amino acid sequences of SEQ ID NO:4 and of SEQ ID NO:5 or mutants of SEQ ID NO:5, preferably C-terminal truncation forms of SEQ ID NO:5 and wherein the N-terminal methionine is preferably cleaved.

In a further preferred embodiment of the present invention, the virus-like particle comprises, consists essentially of, or alternatively consists of, recombinant proteins of Qβ, or fragments thereof, wherein the recombinant proteins comprise, or alternatively consist essentially of, or alternatively consist of mutant Qβ coat proteins. In another preferred embodiment, these mutant coat proteins have been modified by removal of at least one lysine residue by way of substitution, or by addition of at least one lysine residue by way of substitution. Alternatively, these mutant coat proteins have been modified by deletion of at least one lysine residue, or by addition of at least one lysine residue by way of insertion.

Four lysine residues are exposed on the surface of the capsid of Qβ coat protein. Qβ mutants, for which exposed lysine residues are replaced by arginines can also be used for the present invention. The following Qβ coat protein mutants and mutant Qβ VLPs can, thus, be used in the practice of the invention: “Qβ-240” (Lys13-Arg; SEQ ID NO:17), “Qβ-243” (Asn 10-Lys; SEQ ID NO:18), “Qβ-250” (Lys 2-Arg, Lys13-Arg; SEQ ID NO:19), “Qβ-251” (SEQ ID NO:20) and “Qβ-259” (Lys 2-Arg, Lys16-Arg; SEQ ID NO:21). Thus, in further preferred embodiment of the present invention, the virus-like particle comprises, consists essentially of or alternatively consists of recombinant proteins of mutant Qβ coat proteins, which comprise proteins having an amino acid sequence selected from the group of: a) SEQ ID NO:17; b) SEQ ID NO:18; c) SEQ ID NO:19; d) SEQ ID NO:20; and e) SEQ ID NO:21. The construction, expression and purification of the above indicated Qβ coat proteins, mutant Qβ coat protein VLPs and capsids, respectively, are described in WO 02/056905. In particular is hereby referred to Example 18 of above mentioned application.

In a further preferred embodiment of the present invention, the virus-like particle comprises, or alternatively consists essentially of, or alternatively consists of recombinant proteins of Qβ, or fragments thereof, wherein the recombinant proteins comprise, consist essentially of or alternatively consist of a mixture of either one of the foregoing Qβ mutants and the corresponding A1 protein.

In a further preferred embodiment of the present invention, the virus-like particle comprises, or alternatively essentially consists of, or alternatively consists of recombinant proteins, or fragments thereof being capable of assembling into a VLP, of RNA-phage AP205.

In another further preferred embodiment, the virus-like particle comprises, or alternatively consists essentially of, or alternatively consists of coat protein, or fragments thereof being capable of assembling into a VLP, of RNA-phage AP205. AP205 VLPs are highly immunogenic.

WO 2004/007538 describes, in particular in Example 1 and Example 2, how to obtain VLP comprising AP205 coat proteins, and hereby in particular the expression and the purification thereto. WO 2004/007538 is incorporated herein by way of reference. Assembly-competent mutant forms of AP205 VLPs, including AP205 coat protein with the substitution of proline at amino acid 5 to threonine (SEQ ID NO:29), or the substitution of Asparagine at amino acid 14 to Aspartic acid of SEQ ID NO:28 may also be used in the practice of the invention and leads to a further preferred embodiment of the invention.

In a further preferred embodiment of the present invention, the virus-like particle comprises, or alternatively essentially consists of, or alternatively consists of recombinant mutant coat proteins, or fragments thereof, of the RNA-phage AP205.

In a further preferred embodiment of the present invention, the virus-like particle comprises, or alternatively essentially consists of, or alternatively consists of a mixture of recombinant coat proteins, or fragments thereof, of the RNA-phage AP205 and of recombinant mutant coat proteins, or fragments thereof, of the RNA-phage AP205.

In a further preferred embodiment of the present invention, the virus-like particle comprises, or alternatively essentially consists of, or alternatively consists of fragments of recombinant coat proteins or recombinant mutant coat proteins of the RNA-phage AP205.

Recombinant AP205 coat protein fragments capable of assembling into a VLP and a capsid, respectively are also useful in the practice of the invention. These fragments may be generated by deletion, either internally or at the termini of the coat protein and mutant coat protein, respectively. Insertions in the coat protein and mutant coat protein sequence or fusions of a ghrelin-peptide of the invention to the coat protein and mutant coat protein sequence, and compatible with assembly into a VLP, are further embodiments of the invention and lead to chimeric AP205 coat proteins, and particles, respectively. The outcome of insertions, deletions and fusions to the coat protein sequence and whether it is compatible with assembly into a VLP can be determined by electron microscopy.

The crystal structure of several RNA bacteriophages has been determined (Golmohammadi, R. et al., Structure 4:543-554 (1996)). Using such information, surface exposed residues can be identified and, thus, RNA-phage coat proteins can be modified such that one or more reactive amino acid residues can be inserted by way of insertion or substitution. As a consequence, those modified forms of bacteriophage coat proteins can also be used for the present invention. Thus, variants of proteins which form capsids or capsid-like structures (e.g., coat proteins of bacteriophage Qβ, bacteriophage R17, bacteriophage fr, bacteriophage GA, bacteriophage SP, bacteriophage AP205, and bacteriophage MS2) can also be used to prepare modified core particles and preferably modified VLPs and also compositions of the present invention.

Although the sequence of the mutant proteins discussed above will differ from their wild-type counterparts, these mutant proteins will generally retain the ability to form capsids or capsid-like structures. Thus, the invention further includes compositions and vaccine compositions, respectively, which further include mutant of proteins which form capsids or capsid-like structures, as well as methods for preparing such compositions and vaccine compositions, respectively, individual protein subunits used to prepare such compositions, and nucleic acid molecules which encode these protein subunits. Thus, included within the scope of the invention are mutant forms of wild-type proteins which form capsids or capsid-like structures and retain the ability to associate and form capsids or capsid-like structures.

As a result, the invention further includes compositions and vaccine compositions, respectively, comprising proteins, which comprise, or alternatively consist essentially of, or alternatively consist of amino acid sequences which are at least 80%, 85%, 90%, 95%, 97%, or 99% identical to wild-type proteins which form ordered arrays and having an inherent repetitive structure, respectively.

Further included within the scope of the invention are nucleic acid molecules which encode proteins used to prepare compositions of the present invention.

In other embodiments, the invention further includes compositions comprising proteins, which comprise, or alternatively consist essentially of, or alternatively consist of amino acid sequences which are at least 80%, 85%, 90%, 95%, 97%, or 99% identical to any of the amino acid sequences shown in SEQ ID NOs:4-21.

In a further preferred embodiment of the present invention, the at least one ghrelin-peptide of the invention is bound to said core particle and virus-like particle, respectively, by at least one covalent bond. Preferably, the at least one ghrelin-peptide is bound to the core particle and virus-like particle, respectively, by at least one covalent bond, said covalent bond being a non-peptide bond leading to a core particle-ghrelin particle, preferably an ordered and repetitive array and a ghrelin-VLP-conjugate, and preferably array, respectively. This ghrelin-VLP array and conjugate, respectively, has typically and preferably a repetitive and ordered structure since the at least one, but usually more than one, ghrelin-peptide of the invention is bound to the VLP and core particle, respectively, in an oriented manner. Preferably, equal or more than 120, more preferably equal or more than 180, even more preferably equal or more than 270, and again more preferably equal or more than 360 ghrelin-peptides of the invention are bound to the VLP.

In a further preferred embodiment of the present invention, the modified VLP further comprises at least one, preferably one, heterobifunctional cross-linker. The present invention discloses methods of binding of ghrelin-peptide of the invention to core particles and VLPs, respectively. As indicated, in one aspect of the invention, the ghrelin-peptide of the invention is bound to the core particle and VLP, respectively, by way of chemical cross-linking, typically and preferably by using a heterobifunctional cross-linker. Several hetero-bifunctional cross-linkers are known in the art. In preferred embodiments, the hetero-bifunctional cross-linker contains a functional group which can react with preferred first attachment sites, preferably, with the side-chain amino group of lysine residues of the core particle and the VLP or at least one VLP subunit, respectively, and a further functional group which can react with a preferred second attachment site, preferably, a cysteine residue added to or engineered to be added to the ghrelin-peptide of the invention, and optionally also made available for reaction by reduction. Several hetero-bifunctional cross-linkers are known to the art. These include the preferred cross-linkers SMPH (Pierce), Sulfo-MBS, Sulfo-EMCS, Sulfo-GMBS, Sulfo-SIAB, Sulfo-SMPB, Sulfo-SMCC, SVSB, SIA and other cross-linkers available for example from the Pierce Chemical Company (Rockford, Ill., USA), and having one functional group reactive towards amino groups and one functional group reactive towards cysteine residues. Another class of cross-linkers suitable in the practice of the invention is characterized by the introduction of a disulfide linkage between the ghrelin-peptide of the invention and the core particle or VLP upon coupling. Preferred cross-linkers belonging to this class include for example SPDP and Sulfo-LC-SPDP (Pierce).

In an again further preferred embodiment of the present invention, the modified VLP further comprises an amino acid linker, wherein preferably said amino acid linker contains no further ghrelin amino acid residue sequence. In a preferred embodiment of the present invention, the first attachment site comprises, or preferably is, an amino group, preferably the amino group of a lysine residue. In another preferred embodiment of the present invention, the second attachment site comprises, or preferably is, a sulfhydryl group, preferably a sulfhydryl group of a cysteine. In a very preferred embodiment of the invention, the at least one first attachment site is an amino group, preferably an amino group of a lysine residue and the at least one second attachment site is a sulfhydryl group, preferably a sulfhydryl group of a cysteine.

In some embodiments, engineering of an amino acid linker containing a cysteine residue, as a second attachment site or as a part thereof, to the ghrelin-peptide of the invention for coupling to the core particle and VLP, respectively, may be preferred or required. Alternatively, a cysteine may be introduced by addition to the ghrelin-peptide of the invention. Alternatively, the cysteine residue may be introduced by chemical coupling.

The selection of the amino acid linker will be dependent on the nature of the ghrelin-peptide of the invention, on its biochemical properties, such as pI, charge distribution and glycosylation. In general, flexible amino acid linkers are favored. Preferred embodiments of the amino acid linker are selected from the group consisting of: (a) CGG; (b) N-terminal gamma 1-linker; (c) N-terminal gamma 3-linker; (d) Ig hinge regions; (e) N-terminal glycine linkers; (f) (G)kC(G)n with n=0-12 and k=0-5 (SEQ ID NO:34); (g) N-terminal glycine-serine linkers; (h) (G)kC(G)m(S)l(GGGGS)n with n=0-3, k=0-5, m=0-10, l=0-2 (SEQ ID NO:35); (i) GGC; (k) GGC-NH2; (l) C-terminal gamma 1-linker; (m) C-terminal gamma 3-linker; (n) C-terminal glycine linkers; (o) (G)nC(G)k with n=0-12 and k=0-5 (SEQ ID NO:36); (p) C-terminal glycine-serine linkers; (q) (G)m(S)l(GGGGS)n(G)oC(G)k with n=0-3, k=0-5, m=0-10, l=0-2, and o=0-8 (SEQ ID NO:37). In a further preferred embodiment the at least one ghrelin peptide is linked to the VLP via its C-terminal. Therefore C-terminal linkers are preferred embodiments of the invention.

Further preferred examples of amino acid linkers are the hinge region of Immunoglobulins, glycine serine linkers (GGGGS)n (SEQ ID NO:38), and glycine linkers (G)n all further containing a cysteine residue as second attachment site and optionally further glycine residues. Typically preferred examples of said amino acid linkers are N-terminal gammal: CGDKTHTSPP (SEQ ID NO:39); C-terminal gamma 1: DKTHTSPPCG (SEQ ID NO:40); N-terminal gamma 3: CGGPKPSTPPGSSGGAP (SEQ ID NO:41); C-terminal gamma 3: PKPSTPPGSSGGAPGGCG (SEQ ID NO:42); N-terminal glycine linker: GCGGGG (SEQ ID NO:43); C-terminal glycine linker: GGGGCG (SEQ ID NO:44); C-terminal glycine-lysine linker: GGKKGC (SEQ ID NO:45); N-terminal glycine-lysine linker: CGKKGG (SEQ ID NO:46).

In a further preferred embodiment of the present invention, GGCG (SEQ ID NO:47), GGC or GGC-NH2 (“NH2” stands for amidation), GC or C linkers at the C-terminus of the ghrelin peptide are preferred as amino acid linkers. In general, glycine residues will be inserted between bulky amino acids and the cysteine to be used as second attachment site, to avoid potential steric hindrance of the bulkier amino acid in the coupling reaction.

Binding of the ghrelin-peptide of the invention to the core particle and VLP, respectively, by using a hetero-bifunctional cross-linker according to the preferred methods described above, allows coupling of the ghrelin-peptide of the invention to the core particle and the VLP, respectively, in an oriented fashion. Other methods of binding the ghrelin-peptide of the invention to the core particle and the VLP, respectively, include methods wherein the ghrelin-peptide of the invention is cross-linked to the core particle and the VLP, respectively, using the carbodiimide EDC, and NHS. The ghrelin-peptide of the invention may also be first thiolated through reaction, for example with SATA, SATP or iminothiolane. The ghrelin-peptide of the invention, after deprotection if required, may then be coupled to the core particle and the VLP, respectively, as follows. After separation of the excess thiolation reagent, the ghrelin-peptide of the invention is reacted with the core particle and the VLP, respectively, previously activated with a hetero-bifunctional cross-linker comprising a cysteine reactive moiety, and therefore displaying at least one or several functional groups reactive towards cysteine residues, to which the thiolated ghrelin-peptide of the invention can react, such as described above. Optionally, low amounts of a reducing agent are included in the reaction mixture. In further methods, the ghrelin-peptide of the invention is attached to the core particle and the VLP, respectively, using a homo-bifunctional cross-linker such as glutaraldehyde, DSG, BM[PEO]4, BS3, (Pierce Chemical Company, Rockford, Ill., USA) or other known homo-bifunctional cross-linkers with functional groups reactive towards amine groups or carboxyl groups of the core particle and the VLP, respectively. Alternatively the glutamic acid of SEQ ID NO:3 can be used as the second attachment site for linking the ghrelin peptide of the invention to the VLP, preferably through a lysine residue.

Other methods of binding the VLP to a ghrelin-peptide of the invention include methods where the core particle and the VLP, respectively, is biotinylated, and the ghrelin-peptide of the invention expressed as a streptavidin-fusion protein, or methods wherein both the ghrelin-peptides of the invention and the core particle and the VLP, respectively, are biotinylated, for example as described in WO 00/23955. Other ligand-receptor pairs, where a soluble form of the receptor and of the ligand is available, and are capable of being cross-linked to the core particle and the VLP, respectively, or the ghrelin-peptide of the invention, may be used as binding agents for binding the ghrelin-peptide of the invention to the core particle and the VLP, respectively. Alternatively, either the ligand or the receptor may be fused to the ghrelin-peptide of the invention, and so mediate binding to the core particle and the VLP, respectively, chemically bound or fused either to the receptor, or the ligand respectively. Fusion may also be effected by insertion or substitution.

As already indicated, in a preferred embodiment of the present invention, the VLP is the VLP of a RNA phage, and in a more preferred embodiment, the VLP is the VLP of RNA phage Qβ.

One or several antigen molecules, i.e. ghrelin-peptides of the invention, can be attached to one subunit of the capsid or VLP of RNA phages coat proteins, preferably through the exposed lysine residues of the VLP of RNA phages, if sterically allowable. A specific feature of the VLP of the coat protein of RNA phages and in particular of the Qβcoat protein VLP is thus the possibility to couple several antigens per subunit. This allows for the generation of a dense antigen array.

In a preferred embodiment of the invention, the binding of the at least one ghrelin-peptide of the invention to the core particle and the virus-like particle, respectively, is by way of association, between at least one first attachment site of the virus-like particle and at least one second attachment added to the ghrelin-peptide of the invention.

VLPs or capsids of Qβ coat protein display a defined number of lysine residues on their surface, with a defined topology with three lysine residues pointing towards the interior of the capsid and interacting with the RNA, and four other lysine residues exposed to the exterior of the capsid. These defined properties favor the attachment of antigens to the exterior of the particle, rather than to the interior of the particle where the lysine residues interact with RNA.

In very preferred embodiments of the invention, the ghrelin-peptide of the invention is bound via a cysteine residue, having been added to the ghrelin-peptide of the invention, to lysine residues of the VLP of RNA phage coat protein, and in particular to the VLP of Qβ coat protein.

Another advantage of the VLPs derived from RNA phages is their high expression yield in bacteria that allows production of large quantities of material at affordable cost. Another preferred embodiments are VLPs derived from fusion proteins of RNA phage coat proteins with a ghrelin-polypeptide of the invention.

The use of the VLPs as carriers allows the formation of robust antigen arrays and conjugates, respectively, with variable antigen density. In particular, the use of VLPs of RNA phages, and hereby in particular the use of the VLP of RNA phage Qβcoat protein allows achievement of a very high epitope density. The preparation of compositions of VLPs of RNA phage coat proteins with a high epitope density can be effected by using the teaching of this application. In a preferred embodiment of the invention, when a ghrelin-peptide of the invention is coupled to a VLP of a RNA-bacteriophage, preferably to the VLP of Qβcoat protein, an average number of ghrelin-peptide of the invention per subunit of 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4 2.5, 2.6, 2.7, 2.8, 2.9, or higher is preferred.

The non-natural second attachment site, as defined herein, is, when present, a chemical moiety which has been added to the ghrelin-peptide of the invention. Such a non-natural second attachment can be engineered to the ghrelin-peptide of the invention, preferably by genetic engineering or by chemical synthesizing a polypeptide comprising both the ghrelin peptide and the second attachment site.

As described above, four lysine residues are exposed on the surface of the VLP of Qβ coat protein. Typically these residues are derivatized upon reaction with a cross-linker molecule. In the instance where not all of the exposed lysine residues can be coupled to an antigen, the lysine residues which have reacted with the cross-linker are left with a cross-linker molecule attached to the ε-amino group after the derivatization step. This leads to disappearance of one or several positive charges, which may be detrimental to the solubility and stability of the VLP. By replacing some of the lysine residues with arginines, as in the disclosed Qβ coat protein mutants described below, we prevent the excessive disappearance of positive charges since the arginine residues do not react with the preferred cross-linkers. Moreover, replacement of lysine residues by arginines may lead to more defined antigen arrays, as fewer sites are available for reaction to the antigen.

Accordingly, exposed lysine residues were replaced by arginines in the following Qβcoat protein mutants and mutant Qβ VLPs disclosed in this application: Qβ-240 (Lys13-Arg; SEQ ID NO:17), Qβ-250 (Lys 2-Arg, Lys13-Arg; SEQ ID NO:19), Qβ-251; (SEQ ID NO:20) and Qβ-259 (Lys 2-Arg, Lys16-Arg; SEQ ID NO:21). The constructs were cloned, the proteins expressed, the VLPs purified and used for coupling to ghrelin-peptide of the invention.

In a further embodiment, we disclose a Qβ mutant coat protein with one additional lysine residue, suitable for obtaining even higher density arrays of antigens. This mutant Qβcoat protein, Qβ-243 (Asn 10-Lys; SEQ ID NO:18), was cloned, the protein expressed, and the capsid or VLP isolated and purified, showing that introduction of the additional lysine residue is compatible with self-assembly of the subunits to a capsid or VLP.

Prior to the design of a non-natural second attachment site the position at which it should be fused, inserted or generally engineered has to be chosen. Thus, the location of the second attachment site will be selected such that steric hindrance from the second attachment site or any amino acid linker containing the same is avoided. In further embodiments, an antibody response directed at a site distinct from the interaction site of the self-antigen with its natural ligand is desired. In such embodiments, the second attachment site may be selected such that it prevents generation of antibodies against the interaction site of the self-antigen with its natural ligands.

In the most preferred embodiments, the ghrelin-peptide of the invention comprises an added single second attachment site or a single reactive attachment site capable of association with the first attachment sites on the core particle and the VLPs or VLP subunits, respectively. This ensures a defined and uniform binding and association, respectively, of the at least one, but typically more than one, preferably more than 10, 20, 40, 80, 120, 150, 180, 210, 240, 270, 300, 360, 400, 450 ghrelin-peptides of the invention to the core particle and VLP, respectively. The provision of a single second attachment site or a single reactive attachment site on the antigen, thus, ensures a single and uniform type of binding and association, respectively leading to a very highly ordered and repetitive array. For example, if the binding and association, respectively, is effected by way of a lysine- (as the first attachment site) and cysteine- (as a second attachment site) interaction, it is ensured, in accordance with this preferred embodiment of the invention, that only one added cysteine residue per ghrelin-peptide of the invention is capable of binding and associating, respectively, with the VLP and the first attachment site of the core particle, respectively.

In a preferred embodiment, an amino acid linker is bound to the antigen or the antigenic determinant by way of at least one covalent bond, preferably by at least one peptide bond. Preferably, the amino acid linker comprises, or alternatively consists of, the second attachment site. In a further preferred embodiment, the amino acid linker comprises a sulfhydryl group or a cysteine residue. In another preferred embodiment, the amino acid linker is cysteine.

In one preferred embodiment of the invention, the at least one ghrelin-peptide of the invention is fused to the core particle and the VLP, respectively. In again a further preferred embodiment of the invention, the ghrelin-peptide of the invention is fused to at least one subunit of the VLP or of a protein capable of being incorporated into a VLP generating a chimeric VLP-subunit ghrelin-peptide protein fusion. Gene encoding ghrelin-peptide of the invention, either internally or preferably to the N- or the C-terminus to the gene encoding the coat protein of the VLP. Fusion may also be effected by inserting sequences of the ghrelin-peptide into a mutant of a coat protein where part of the coat protein sequence has been deleted, that are further referred to as truncation mutants. Truncation mutants may have N- or C-terminal, or internal deletions of part of the sequence of the coat protein. For example for the specific VLP HBcAg, amino acids 79-80 are replaced with a foreign epitope. The fusion protein shall preferably retain the ability of assembly into a VLP upon expression which can be examined by electromicroscopy.

Flanking amino acid residues may be added to increase the distance between the coat protein and foreign epitope. Glycine and serine residues are particularly favored amino acids to be used in the flanking sequences. Such a flanking sequence confers additional flexibility, which may diminish the potential destabilizing effect of fusing a foreign sequence into the sequence of a VLP subunit and diminish the interference with the assembly by the presence of the foreign epitope.

In one preferred embodiment, the modified VLP is a mosaic VLP. In a preferred embodiment, said mosaic VLP comprises or alternatively consists of at least one fusion protein and at least one viral coat protein. In other embodiments, the at least one ghrelin-peptide of the invention can be fused to a number of other viral coat protein, as way of examples, to the C-terminus of a truncated form of the A1 protein of Qβ (Kozlovska, T. M., et al., Intervirology 39:9-15 (1996)), or being inserted between position 72 and 73 of the CP extension. For example, Kozlovska et al., (Intervirology, 39: 9-15 (1996)) describe QβA1 protein fusions where the epitope is fused at the C-terminus of the QβCP extension truncated at position 19. As another example, the ghrelin-peptide can be inserted between amino acid 2 and 3 of the fr CP, leading to a ghrelin-peptide-fr CP fusion protein (Pushko P. et al., Prot. Eng. 6:883-891 (1993)). Furthermore, ghrelin-peptide can be fused to the N-terminal protuberant β-hairpin of the coat protein of RNA phage MS-2 (WO 92/13081). Alternatively, ghrelin-peptide can be fused to a capsid protein of papillomavirus, preferably to the major capsid protein L1 of bovine papillomavirus type 1 (BPV-1) (Chackerian, B. et al., Proc. Natl. Acad. Sci. USA 96:2373-2378 (1999), WO 00/23955). Substitution of amino acids 130-136 of BPV-1 L1 with a ghrelin-peptide is also an embodiment of the invention. Further embodiments of using antigen of the invention to coat protein, mutants or fragments thereof, to a coat protein of a virus have been disclosed in WO 2004/009124 page 62 line 20 to page 68 line 17 and herein are incorporated by way of reference.

In a further very preferred embodiment of the invention, the ghrelin-peptide of the invention is a ghrelin-peptide with a length of 6 or 8 amino acid residues which peptide is homologous to SEQ ID NO:1 or SEQ ID NO:3 and is selected from the group consisting of a) human ghrelin; b) cat ghrelin; c) dog ghrelin; d) bovine ghrelin; e) sheep ghrelin; f) horse ghrelin, and g) pig ghrelin.

In a further very preferred embodiment of the invention, the ghrelin-peptide of the invention is the human, cat, pig, horse, sheep, bovine, guinea pig, dog or mouse ghrelin-peptide of the invention, respectively. Ghrelin-peptides of the invention can be produced by recombinantly expression of DNA encoding ghrelin-peptide of the invention. Preferably, the DNA does not encode the preproghrelin but only the peptidic backbone of the active n-octanoyl-modified peptide. Various examples hereto have been described in the literature and can be used, possibly after modifications, to express ghrelin-peptide of the invention of any desired species, preferably in the context of fusion polypeptides, e.g. a fusion with GST, DHFR, histdine tag, the Flag tag, myc tag or the constant region of an antibody (Fc region). By introducing an enterokinase cleavage site between the ghrelin-peptide of the invention and the tag, the ghrelin-peptide of the invention can be separated from the tag after purification by digestion with enterokinase. In another approach the ghrelin-peptide of the invention can be synthesized in vitro with or without n-octanoyl-modification using standard peptide synthesis reactions known to a person skilled in the art.

In one preferred embodiment, the ghrelin-peptide is selected from the GSSFLS (SEQ ID NO:1) or GSSFLSPE (SEQ ID NO:3). In one further preferred embodiment, the ghrelin-peptide differs at only 1 position from SEQ ID NO:1 or SEQ ID NO:3, wherein said difference does not result in SEQ ID NO:2. In one still further preferred embodiment, said difference is a difference in amino acid nature at a particular position, but not a difference in length.

In one preferred embodiment, the ghrelin-peptide does not contain a n-octanoyl-modification.

Since ghrelins of various species are highly homologous, it is likely that cross-reactive antibody responses can be induced. Thus, antibody responses against dog or mouse ghrelin may also recognize human ghrelin and vice versa. It is therefore within the scope of this invention that all ghrelin-peptides of the invention with amino acid identities > than 80%, preferably higher than 85%, more preferably higher than 90%, or even more preferably higher than 95%, 97% or even 99% to human ghrelin may be used for vaccination and vice versa. Such ghrelin-peptides of the invention which differ at only one position from SEQ ID NOs 1 or 3, and which are not SEQ ID NO:2, are preferred ghrelin-peptides of the invention.

In one preferred embodiment of the present invention, the modified VLP further comprises at least one polypeptide, wherein said at least one polypeptide is fused to the ghrelin-peptide of the invention. The fusion of additional amino acid sequence to the ghrelin-peptide may increase the solubility and/or the stability of the ghrelin-peptide. Typically and pereferably said at least one polyeptide does not comprise or consists of a amino acid sequence derived from a ghrelin polypeptide. In one further preferred embodiment, said at least one polyeptide is an amino acid linker. In a preferred embodiment, said amino acid linker comprises or alternatively consists of at least one second attachment site. In a further preferred embodiment, said amino acid linker of the invention comprises or alternatively or preferably consists of a linker sequence of C, GC, or GGC. Preferably said amino acid linker is fused to the C-terminus of the ghrelin of the invention. Preferably, the ghrelin-peptide of the invention with said added at least one second attachment site comprises, or alternatively consists of an amino acid sequence selected from the group consisting of

| | | |
|---|---|---|
| Ghrel24-31GC | GSSFLSPEGC | (SEQ ID NO:50) |
| Ghrel24-31C | GSSFLSPEC | (SEQ ID) NO:51) |
| Ghrel24-3OGC | GSSFLSPGC | (SEQ ID NO:52) |
| Ghrel24-30C | GSSFLSPC | (SEQ ID NO:53) |
| Ghrel24-29GC | GSSFLSGC | (SEQ ID NO:54) |
| Ghrel24-29C | GSSFLSC | (SEQ ID NO:55) |

In a further very preferred embodiment of the present invention, the ghrelin-peptide of the invention with said at least one second attachment site comprises, or alternatively consists of an amino acid sequence of SEQ ID NO:50 or SEQ ID NO:51.

Some of the very preferred ghrelin-peptides of the invention are described in EXAMPLE 9. These peptides comprise an C-terminal cysteine residue as a second attachment added for coupling to VLPs and Pili. These very preferred short ghrelin-peptides of the invention are capable of having a very enhanced immunogenicity when coupled to VLP and to a core particle, respectively. The preferred ghrelin-peptides of the invention are, furthermore, capable of also overcoming safety issues that arise when targeting self-proteins as shorter fragment are much less likely to contain T cell epitopes. Typically, the shorter the peptides, the safer with respect to T cell activation. However, too short peptides, i.e. having less than 4 amino acids, may fail to induce high-affinity antibodies that are able to strongly bind ghrelin in solution.

The very preferred ghrelin-peptide fragment corresponding to the ghrelin fragment of residue 1-8 (GSSFLSPE) (SEQ ID NO:3) was chosen primarily because the N-terminal segment of ghrelin is identical among all known species. Additionally, it is likely to be identical in species where ghrelin has yet to be identified. Furthermore, the C-terminal residue, a glutamate, would enhance solubility, facilitating the production of a soluble vaccine product when coupled to VLP and to a core particle, respectively. In fact, the solubility of peptides is often a limiting factor for coupling efficiency and vaccine stability. Further reasoning includes avoiding a potential T cell epitope. The choice of a smaller peptide fragment reduces the probability of a T cell epitope being present. Coupling ghrelin residues 1-8 via the C-terminus to VLP will induce N-terminal specific antibodies when immunized into mice that are capable of binding active ghrelin and hence, preferably, prevent it's passing of the blood brain barrier, leading to reduced food intake.

The further very preferred ghrelin-peptide fragments corresponding to the murine ghrelin-peptide fragment of residues 1-6 (GSSFLS) (SEQ ID NO:1) has been chosen for similar reasons to that above. Coupling ghrelin residues 1-6 via its C-terminus to VLP will induce antibodies capable of neutralizing active ghrelin and, hence preferably, prevent it's passing of the blood brain barrier, leading to reduced food intake.

In one further aspect, the invention provides a composition comprising the modified core particle, or preferably modified VLP, of the invention.

In still one aspect, the invention provides a pharmaceutical composition comprising the modified core particle, or preferably modified VLP, of the invention and an acceptable pharmaceutical carrier.

In still one aspect, the invention provides a vaccine composition comprising the modified core particle or preferably modified VLP, of the invention.

In one embodiment, the invention provides a vaccine composition of the invention further comprising an adjuvant. In another embodiment, the vaccine composition of the invention is devoid of an adjuvant. In a further embodiment of the invention, the vaccine composition comprises a core particle of the invention, wherein the core particle comprises, preferably is, a virus-like particle, wherein preferably said virus-like particle is a recombinant virus-like particle. Preferably, the virus-like particle comprises, or alternatively consists essentially of, or alternatively consists of, recombinant proteins, or fragments thereof, of a RNA-phage, preferably of coat proteins of RNA phages. In a preferred embodiment, the coat protein of the RNA phages has an amino acid are selected from the group consisting of: (a) SEQ ID NO:4; (b) a mixture of SEQ ID NO:4 and SEQ ID NO:5; (c) SEQ ID NO:6; (d) SEQ ID NO:7; (e) SEQ ID NO:8; (f) SEQ ID NO:9; (g) a mixture of SEQ ID NO:9 and SEQ ID NO:10; (h) SEQ ID NO:11; (i) SEQ ID NO:12; (k) SEQ ID NO:13; (l) SEQ ID NO:14; (m) SEQ ID NO:15; (n) SEQ ID NO:16; and (o) SEQ ID NO:28. Alternatively, the recombinant proteins of the virus-like particle of the vaccine composition of the invention comprise, or alternatively consist essentially of, or alternatively consist of mutant coat proteins of RNA phages, wherein the RNA-phage is selected from the group consisting of: (a) bacteriophage Qβ; (b) bacteriophage R17; (c) bacteriophage fr; (d) bacteriophage GA; (e) bacteriophage SP; (f) bacteriophage MS2; (g) bacteriophage M11; (h) bacteriophage MX1; (i) bacteriophage NL95; (k) bacteriophage f2; (l) bacteriophage PP7; and (m) bacteriophage AP205.

In a preferred embodiment, the mutant coat proteins of said RNA phage have been modified by removal, or by addition of at least one lysine residue by way of substitution. In another preferred embodiment, the mutant coat proteins of said RNA phage have been modified by deletion of at least one lysine residue or by addition of at least one lysine residue by way of insertion. In a preferred embodiment, the virus-like particle comprises recombinant proteins or fragments thereof, of RNA-phage Qβ or alternatively of RNA-phage fr, of RNA-phage GA or of RNA-phage AP205.

In one further aspect, the invention provides a method of immunization against ghrelin, preferably for treating obesity, comprising the step of administering the vaccine composition of the invention to an animal or to a human. In one preferred embodiment, the vaccine composition is administered to a human and wherein said ghrelin-peptide is a human ghrelin-peptide.

In another preferred embodiment, said animal is of feline origin and wherein said ghrelin-peptide is a feline ghrelin-peptide. In still another preferred embodiment, said animal is of canine origin and wherein said ghrelin-peptide is a canine ghrelin-peptide.

In one aspect, the invention provides a modified core particle, preferably modified VLP, of the invention, for use as a medicament.

In one further aspect, the invention provides a use of the modified core particle, preferably VLP, of the invention, for the manufacture of a medicament for treatment of obesity.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

### EXAMPLES

### Example 1

### Construction of HBcAg1-185-Lys

Hepatitis core Antigen (HBcAg) 1-185 was modified as described in Example 23 of WO 02/056905. A part of the c/e1 epitope (residues 72 to 88) region (Proline 79 and Alanine 80) was genetically replaced by the peptide Gly-Gly-Lys-Gly-Gly (SEQ ID NO:33), resulting in the HBcAg-Lys construct (SEQ ID NO:26). The introduced Lysine residue contains a reactive amino group in its side chain that can be used for intermolecular chemical crosslinking of HBcAg particles with any antigen containing a free cysteine group. PCR methods and conventional cloning techniques were used to prepare the HBcAg1-185-Lys gene.

The Gly-Gly-Lys-Gly-Gly sequence (SEQ ID NO:33) was inserted by amplifying two separate fragments of the HBcAg gene from pEco63, as described above in Example 23 of WO 02/056905 and subsequently fusing the two fragments by PCR to assemble the full length gene. The following PCR primer combinations were used:

fragment 1:
Primer 1: EcoRIHBcAg(s)

(SEQ ID NO:56)

Primer 2: Lys-HBcAg(as)

(SEQ ID NO:57)

fragment 2:
Primer 3: Lys-HBcAg(s)

(SEQ ID NO:58)

Primer 4: HBcAgwtHindIIII

(SEQ ID NO:59)

CGCGTCCCAAGCTTCTAACATTGAGATTCCCGAGATTG
Assembly:
Primer 1: EcoRIHBcAg(s)

(SEQ ID NO:56)

Primer 2: HBcAgwtHindIIII

(SEQ ID) NO:59)

The assembled full length gene was then digested with the EcoRI (GAATTC) and HindIII (AAGCTT) enzymes and cloned into the pKK vector (Pharmacia) cut at the same restriction sites.

### Example 2

### Fusion of a Peptide Epitope in the MIR Region of HbcAg

The residues 79 and 80 of HBcAg1-185 were substituted with the epitope CεH3 of sequence VNLTWSRASG (SEQ ID NO:60). The CεH3 sequence stems from the sequence of the third constant domain of the heavy chain of human IgE. The epitope was inserted in the HBcAg1-185 sequence using an assembly PCR method. In the first PCR step, the HBcAg1-185 gene originating from ATCC clone pEco63 and amplified with primers HBcAg-wt EcoRI fwd and HBcAg-wt Hind III rev was used as template in two separate reactions to amplify two fragments containing sequence elements coding for the CεH3 sequence. These two fragments were then assembled in a second PCR step, in an assembly PCR reaction.

Primer combinations in the first PCR step: CεH3fwd with HBcAg-wt Hind III rev, and HBcAg-wt EcoRI fwd with CεH3rev. In the assembly PCR reaction, the two fragments isolated in the first PCR step were first assembled during 3 PCR cycles without outer primers, which were added afterwards to the reaction mixture for the next 25 cycles. Outer primers: HBcAg-wt EcoRI fwd and HBcAg-wt Hind III rev.

The PCR product was cloned in the pKK223.3 using the EcoRI and HindIII sites, for expression in E. coli (see Example 23 of WO 02/056905). The chimeric VLP was expressed in E. coli and purified as described in Example 23 of WO 02/056905. The elution volume at which the HBcAg1-185-CεH3 eluted from the gel filtration showed assembly of the fusion proteins to a chimeric VLP.

Primer sequences:

CεH3 fwd:

(SEQ ID NO:61)

5′GTT AAC TTG ACC TGG TCT CGT GCT TCT GGT GCA TCC
AGG GAT CTA GTA GTC 3′

(SEQ ID NO:62)

V N L T W S R A S G A80 S R D L V V86
C□H3rev:

(SEQ ID NO:63)

5′ACC AGA AGC ACG AGA CCA GGT CAA GTT AAC ATC TTC
CAA ATT ATT ACC CAC 3′

(SEQ ID NO:64)

D78 E L N N G V72
HBcAg-wt EcoRI fwd:

(SEQ ID NO:65)

5′CCGgaattcATGGACATTGACCCTTATAAAG
HBcAg-wt Hind III rev:

(SEQ ID NO:66)

5′CGCGTCCCaagcttCTAACATTGAGATTCCCGAGATTG

### Example 3

### Fusion of the Ghrelin 24-31-Peptide Epitope in the MIR Region of HbcAg

The residues 79 and 80 of HBcAg1-185 are substituted with the ghrelin-peptide epitope of sequence: GSSFLSPE (SEQ ID NO:3). Two overlapping primers are designed using the same strategy described in Example 2, and the fusion protein constructed by assembly PCR. The PCR product is cloned in the pKK223.3 vector, and expressed in E. coli K802. The chimeric VLPs are expressed and purified as described in Example 24 of WO 02/056905.

### Example 4

### Fusion of the Ghrelin 24-31-Peptide Epitope to the C-Terminus of the Qβ A1 Protein Truncated at Position 19 of the CP Extension

A primer annealing to the 5′ end of the QβA1 gene and a primer annealing to the 3′ end of the A1 gene and comprising additionally a sequence element coding a ghrelin fragment, of sequence GSSFLSPE (SEQ ID NO:3), are used in a PCR reaction with p Qβ10 as template. The PCR product is cloned in p Qβ10 (Kozlovska T. M. et al., Gene 137: 133-37 (1993)), and the chimeric VLP expressed and purified as described in Example 18 of WO 02/056905.

### Example 5

### Insertion of the Ghrelin 24-31-Peptide Epitope Between Positions 2 and 3 of fr Coat Protein

Complementary primers coding for the sequence of the ghrelin-peptide of sequence GSSFLSPE (SEQ ID NO:3), and containing Bsp119I compatible ends and additional nucleotides enabling in frame insertion, are inserted in the Bsp119I site of the pFrd8 vector (Pushko, P. et al., Prot. Eng. 6: 883-91 (1993)) by standard molecular biology techniques. Alternatively, the overhangs of the pFrd8 vector are filled in with Klenow after digestion with Bsp119I, and oligonucleotides coding for the sequence of the murine ghrelin-peptide and additional nucleotides for in frame cloning are ligated in pFrd8 after the Klenow treatment. Clones with the insert in the right orientation are analyzed by sequencing. Expression and purification of the chimeric fusion protein in E. coli JM109 or E. coli K802 is performed as described in Pushko, P. et al, Prot. Eng. 6:883-91 (1993), but for the chromatography steps which are performed using a Sepharose CL-4B or Sephacryl S-400 (Pharmacia). The cell lysate is precipitated with ammonium sulphate, and purified by two successive gel filtration purification steps, similarly to the procedure described for Qβ in Example 18 of WO 02/056905.

### Example 6

### Insertion of the Ghrelin 24-31-Peptide Epitope Between Positions 67 and 68 of Ty1 Protein p1 in the Vector pOGS8111

Two complementary oligonucleotides coding for the human ghrelin-peptide sequence GSSFLSPE (SEQ ID NO:3), with ends compatible with the NheI site of pOGS8111 are synthesized. Additional nucleotides are added to allow for in frame insertion of a sequence coding for the murine ghrelin epitope according to the description of EP06777111. The amino acids AS and SS flanking the inserted epitope are encoded by the altered NheI sites resulting from the insertion of the oligonucleotide in the TyA(d) gene of pOGS8111.

POGS8111 is transformed into S. cervisiae strain MC2, for expression of the chimeric Ty VLP as described in EP0677111 and references therein. The chimeric Ty VLP is purified by sucrose gradient ultracentrifugation as described in EP0677111.

### Example 7

### Insertion of the Ghrelin 24-31-Peptide Epitope in to the Major Capsid Protein Li of Papillomavirus Type 1 (BPV-1)

A sequence coding for the ghrelin epitope having the sequence GSSFLSPE (SEQ ID NO:3) is substituted to the sequence coding for amino acids 130-136 of the BPV-1 Li gene cloned in the pFastBac1 (GIBCO/BRL) vector as described (Chackerian, B. et al., Proc. Natl. Acad. USA 96: 2373-2378 (1999)). The sequence of the construct is verified by nucleotide sequence analysis. Recombinant baculovirus is generated using the GIBCO/BRL baculovirus system as described by the manufacturer. The chimeric VLPs are purified from baculovirus infected Sf9 cells as described by Kimbauer, R. et al., Proc. Natl. Acad. Sci. 89:12180-84 (1992) and Greenstone, H. L., et al., Proc. Natl. Acad. Sci. 95:1800-05 (1998).

### Example 8

### Immunization of Mice with Ghrelin-Peptides Fused to VLPs

Chimeric VLPs displaying the murine ghrelin epitope of sequence GSSFLSPE (SEQ ID NO:3) generated in Examples 3, 5, 6 and 7 are used for immunization of mice as described in Example 12. The sera obtained from the immunized mice are analyzed in a ghrelin-specific ELISA as described in Example 13. The effect of the vaccine is examined by following the weight increase of the mice and by measuring food uptake.

### Example 9

### Coupling of Ghrelin-Peptides to VLPs

Ghrelin fragments 24-31 and 24-30, including a GC or C linker sequence fused to the C-terminus of the ghrelin fragments (SEQ ID NO: 50-53) were chemically synthesized according to standard procedures.

Ghrelin fragments 24-29, including a GC or C linker sequence fused to the C-terminus of the ghrelin fragment, (SEQ ID NO:54-55) are chemically synthesized according to standard procedures.

| | | |
|---|---|---|
| Ghrel24-31GC | GSSFLSPEGC | (SEQ ID NO:50) |
| Ghrel24-31C | GSSFLSPEC | (SEQ ID NO:51) |
| Ghrel24-30GC | GSSFLSPGC | (SEQ ID NO:52) |
| Ghrel24-30C | GSSFLSPC | (SEQ ID NO:53) |
| Ghrel24-29GC | GSSFLSGC | (SEQ ID NO:54) |
| Ghrel24-29C | GSSFLSC | (SEQ ID NO:55) |

A solution of 2 ml of 2.0 mg/ml Qβ VLP in 20 mM Hepes, 150 mM NaCl pH 7.2 was reacted for 30 minutes with 114.4 μl of a SMPH (Pierce) solution (from a 50 mM stock solution dissolved in DMSO) at 25° C. The reaction solution was subsequently dialyzed twice for 2 hours against 2 L of 20 mM Hepes, 150 mM NaCl, pH 7.2 at 4° C.

The dialysed, derivatized Qβ VLP was subsequently used to couple ghrelin 24-31-GC, ghrelin 24-31C, ghrelin 24-30GC or ghrelin 24-30C. Briefly, 1 ml of derivatized Qβ VLP (at a concentration of 2 mg/ml) was reacted with 286 μl of a 10 mM peptide solution for 2 hours at 15° C. in 20 mM Hepes, 150 mM NaCl, pH 7.2. The coupling reactions were then centrifuged at 13 000 rpm for 5 minutes and the supernatants were collected and dialyzed once for 2 hours and then overnight against 2 L of 20 mM Hepes, 150 mM NaCl, pH 7.2 at 4° C.

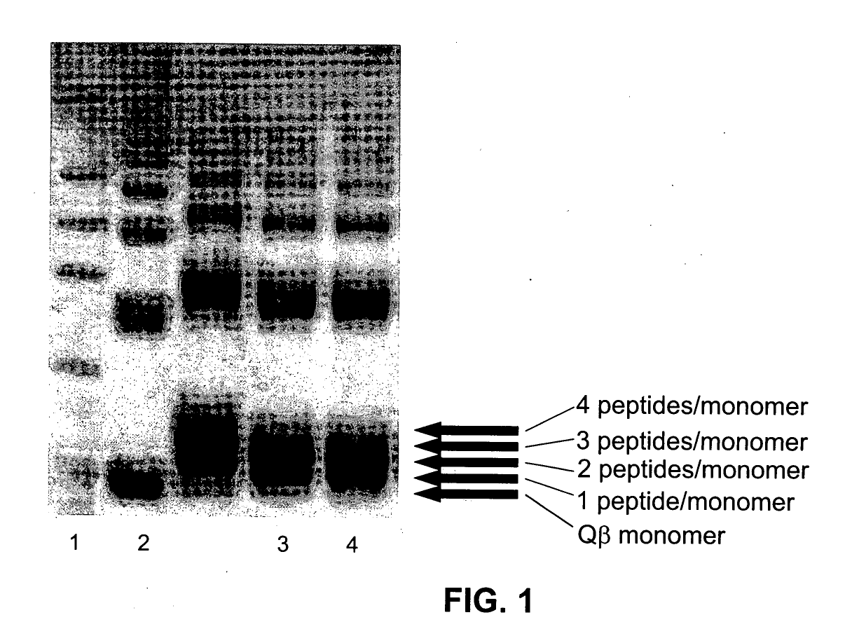

The covalent chemical coupling of ghrelin peptides to the Qβ VLP was assessed by SDS-PAGE using 16% TRIS/BIS SDS-PAGE gels (Invitrogen). Coomassie blue stained gels of the coupling reaction demonstrated the appearance of bands with molecular weights corresponding to those predicted for ghrelin peptides covalently linked to Qβ (FIG. 1). Coupling bands corresponding to one, two, three or four peptides coupled per subunit are indicated by arrows. The appearance of these additional bands as compared to derivatized Qβ VLP alone, demonstrates, that the ghrelin fragments were covalently coupled to Qβ VLP. The coupling efficiency [i.e. mol Qβ-ghrelin/mol Qβ monomer (total)] was estimated, by densitometric analysis of the Coomassie blue stained SDS-PAGE, to be approximately 2 ghrelin fragments per Qβ monomer.

The dialysed, derivatized Qβ VLP is subsequently used to couple ghrelin 24-29-GC or ghrelin 24-29-C. Briefly, 1 ml of derivatized Qβ VLP (at a concentration of 2 mg/ml) is reacted with 286 μl of a 10 mM peptide solution for 2 hours at 15° C. in 20 mM Hepes, 150 mM NaCl, pH 7.2. The coupling reactions are then centrifuged at 13 000 rpm for 5 minutes and the supernatants are collected and dialyzed once for 2 hours and then overnight against 2 L of 20 mM Hepes, 150 mM NaCl, pH 7.2 at 4° C.

Coupling of Ghrelin-Peptides to fr VLP

A solution of 120 μM fr VLP in 20 mM Hepes, 150 mM NaCl pH 7.2 is reacted for 30 minutes with a 10 fold molar excess of SMPH (Pierce)), diluted from a stock solution in DMSO, at 25° C. on a rocking shaker. The reaction solution is subsequently dialyzed twice for 2 hours against 1 L of 20 mM Hepes, 150 mM NaCl, pH 7.2 at 4° C. The dialyzed fr reaction mixture is then reacted with equimolar concentration of ghrelin-peptide or a ration of 1:2 ghrelin-peptide/fr over night at 16° C. on a rocking shaker. Coupling products are analysed by SDS-PAGE.

Coupling of Ghrelin-Peptides to AP205 VLP

A solution of 120 μM AP205 VLP in 20 mM Hepes, 150 mM NaCl pH 7.2 is reacted for 30 minutes with a 10 fold molar excess of SMPH (Pierce)), diluted from a stock solution in DMSO, at 25° C. on a rocking shaker. The reaction solution is subsequently dialyzed twice for 2 hours against 1 L of 20 mM Hepes, 150 mM NaCl, pH 7.2 at 4° C. The dialyzed AP205 reaction mixture is then reacted with equimolar concentration of ghrelin-peptide or a ration of 1:2 ghrelin-peptide/AP205 over night at 16° C. on a rocking shaker. Coupling products are analysed by SDS-PAGE.

Coupling of Ghrelin-Peptides to HBcAg-Lys-2cys-Mut

A solution of 120 μM HBcAg-Lys-2cys-Mut in 20 mM Hepes, 150 mM NaCl pH 7.2 is reacted for 30 minutes with a 10 fold molar excess of SMPH (Pierce), diluted from a stock solution in DMSO, at 25° C. on a rocking shaker. The reaction solution is subsequently dialyzed twice for 2 hours against 1 L of 20 mM Hepes, 150 mM NaCl, pH 7.2 at 4° C. The dialyzed HBcAg-Lys-2cys-Mut reaction mixture is then reacted with equimolar concentration of ghrelin-peptide or a ration of 1:2 ghrelin-peptide/HBcAg-Lys-2cys-Mut over night at 16° C. on a rocking shaker.

Coupling Products are Analysed by SDS-PAGE.

Coupling of ghrelin-peptides to Pili

A solution of 125 μM Type-1 pili of E. coli in 20 mM Hepes, pH 7.2, is reacted for 60 minutes with a 50-fold molar excess of cross-linker SMPH (Pierce), diluted from a stock solution in DMSO, at RT on a rocking shaker. The reaction mixture is desalted on a PD-10 column (Amersham-Pharmacia Biotech). The protein-containing fractions eluating from the column are pooled, and the desalted derivatized pili protein is reacted with the ghrelin-peptides in equimolar or in a ratio of 1:2 peptide pili over night at 16° C. on a rocking shaker. Coupling products are analysed by SDS-PAGE.

### Example 10

### Immunization of Mice with Ghrelin 24-31-GC, 24-31C, 24-30GC or 24-30C Coupled to Qβ□VLP

Adult female, C57BL/6 mice (5 per group) were vaccinated with murine ghrelin 24-31-GC, 24-31C, 24-30GC or 24-30C coupled to the prior art Qβ VLP (obtained in EXAMPLE 9a). 100 μg of dialyzed vaccine from each sample were diluted in PBS to a volume of 200 μl and injected subcutaneously (100 μl on two ventral sides) on days 0, 14, 28 and 42. The vaccine was administered without adjuvant. As a control, a group of mice was injected with PBS. Mice were bled retro-orbitally on day 0, 14, 28, 42 and 56 and their sera analyzed by ELISA as described in EXAMPLE 11.

### Example 11

### Detection of Ghrelin-Specific Antibodies in an ELISA

ELISA plates (96 well MAXIsorp, NUNC immuno plate) were coated with ghrelin protein (Bachem) at a concentration of 20 μg/ml in coating buffer (0.1 M NaHCO3, pH 9.6), over night at 4° C. After washing the plates in wash buffer (PBS-0.05% Tween), the plates were blocked with blocking buffer (2% BSA-PBS-Tween 20 solution) for 2 h at 37° C. and then washed again and incubated with serially diluted mouse sera. As a control, pre-immune serum of the same mice was also tested. Plates were incubated at RT for 2 h. After further washing, bound antibodies were detected with a HRPO-labeled, Fc specific, goat anti-mouse IgG antibody (Jackson Immunoresearch) and incubated for 1 h at RT. After further washing, plates were developed with OPD solution (1 OPD tablet, 25 ul OPD buffer and 8 ul H₂O₂) for 6 minutes and the reaction was stopped with 5% H₂SO₄ solution. Plates were read at 450 nm on an ELISA reader (Biorad Benchmark). ELISA titers are expressed as serum dilutions which lead to half maximal OD in the ELISA assay. TABLE 1 shows the average titers of ghrelin-specific antibodies. Results shown are titres from pooled sera of 5 mice per group. ELISA titers are expressed as serum dilutions which lead to half maximal OD in the ELISA assay. All mice immunized with murine ghrelin 24-31-GC, 24-31C, 24-30GC or 24-30C coupled to the prior art Qβ VLP, elicited good ghrelin-specific antibody titers by day 56 (TABLE 1). Pre-immune sera or sera from mice injected with PBS did not show any reactivity against the murine ghrelin peptide. The half maximal OD titer was less than 100, which was considered to be below the cut-off of the assay. This clearly demonstrates that a ghrelin-VLP conjugate is able to induce a high antibody titer against ghrelin protein, even if it is a self protein. This clearly indicates that antibodies raised with ghrelin fragments were able to recognize ghrelin protein.

TABLE 1
Average anti-ghrelin-specific IgG antibody titer (expressed
as a dilution factor) in mice immunized with Qb-Ghr 24-31GC,
Qb-Ghr 24-31C, Qb-Ghr 24-30GC or Qb-Ghr 24-30C on
day 0, 14, 28 and 42.

Days after first immunization

| Immunization | day 14 | day 28 | day 42 | day 56 |
|---|---|---|---|---|
| Qb-Ghr 24-31 GC | 2663 | 10978 | 16416 | 43117 |
| Qb-Ghr 24-31 C | 2196 | 9346 | 14549 | 69877 |
| Qb-Ghr 24-30 GC | 3283 | 4989 | 17507 | 64474 |
| Qb-Ghr 24-30 C | 3283 | 11898 | 36707 | 15147 |
| PBS | 100 | 100 | 100 | 100 |

### Example 12

### Efficacy Experiments with Ghrelin 24-31 GC, Ghrelin 24-31C and Ghrelin 24-30GC Coupled to Qβ VLP in a Diet Induced Animal Model of Obesity

Adult female, C57BL/6 mice (5 per group) with comparable starting weights (18.71 g-19.75 g) were vaccinated, as described in EXAMPLE 10 with either ghrelin 24-31GC, ghrelin 24-31C and ghrelin 24-30GC coupled to Qβ VLP, obtained in EXAMPLE 9. As a control, mice were injected with PBS. All mice were placed on a high fat diet (35% fat by weight, 60% as energy) after the first injection, in order to facilitate the development of diet-induced obesity. Food and water were administered ad libitum. The body weights of individual animals were monitored in regular intervals over a period of approximately 90 days after the first injection.

As shown in TABLE 2, mice immunized with ghrelin 24-31GC, ghrelin 24-31C and ghrelin 24-30GC coupled to Qβ VLP gained less weight in the course of the experiment than the control animals, which had been injected with PBS. In fact, 88 days after the first immunisation the control animals had increased their weight by roughly 76% whereas ghrelin 24-31GC, ghrelin 24-31C and ghrelin 24-30GC coupled to Qβ VLP immunised mice had only increased their weight by 60%, 67% and 73%, respectively. Hence, these three vaccinated groups displayed a clearly reduced weight gain compared to control groups. These results clearly demonstrate that a ghrelin-VLP conjugate is able to reduce body weight gain.

TABLE 2
Average body weight gain and SEM, expressed as percent, of
10 mice per group immunized with ghrelin 24-31GC, ghrelin
24-31C and ghrelin 24-30GC coupled to Qβ VLP over 88 days.

| | d 14 | d 22 | d 29 | d 36 | d 42 | d 51 | d 56 | d 65 | d 78 | d 88 |
|---|---|---|---|---|---|---|---|---|---|---|
| | Body weight gain (%) | | | | | | | | | |
| Qb-Ghr 24-31GC | 11.42 | 18.31 | 18.15 | 20.39 | 25.20 | 33.00 | 39.79 | 43.06 | 62.82 | 60.18 |
| Qb-Ghr 24-31C | 10.77 | 17.05 | 15.79 | 19.72 | 23.70 | 31.59 | 39.60 | 44.89 | 65.62 | 67.05 |
| Qb-Ghr 24-30GC | 12.92 | 16.36 | 15.20 | 24.73 | 29.30 | 35.81 | 42.50 | 45.47 | 62.91 | 73.33 |
| PBS | 14.70 | 18.45 | 24.32 | 26.50 | 32.73 | 39.57 | 47.70 | 54.75 | 71.48 | 76.05 |
| | SEM (%) | | | | | | | | | |
| Qb-Ghr 24-31GC | 0.78 | 0.70 | 2.11 | 1.35 | 1.48 | 1.99 | 2.59 | 3.31 | 5.68 | 4.55 |
| Qb-Ghr 24-31C | 1.37 | 2.12 | 1.69 | 1.61 | 3.27 | 3.87 | 4.58 | 4.79 | 5.91 | 5.27 |
| Qb-Ghr 24-30GC | 1.24 | 1.70 | 1.48 | 2.35 | 3.85 | 3.21 | 3.90 | 4.84 | 6.12 | 5.76 |
| PBS | 1.37 | 1.76 | 2.35 | 2.87 | 4.40 | 5.73 | 6.70 | 8.17 | 9.94 | 11.73 |

### Example 13

### Immunization of Mice with Ghrelin 24-29-GC and 24-29C Coupled to the Qβ VLP

Adult, male or female, C57BL/6 mice are vaccinated with ghrelin 24-29-GC and 24-29C coupled to Qβ VLP, obtained in EXAMPLE 9. Briefly, 100 μg of dialyzed vaccine from each sample is diluted in PBS to a volume of 200 μl and injected subcutaneously (100 μl on two ventral sides) on days 0, 14, 28 and 42 and subsequently as required. The vaccine is administered with or without adjuvant. As a control, a group of mice are immunized with Qβ VLP or injected with PBS, with or without adjuvant. Mice are bled retro-orbitally on day 0, 14, 28, 42 and subsequently at regular intervals. The ghrelin specific antibodies are then quantified by ELISA as described in EXAMPLE 11.

### Example 14

### Efficacy Experiments with Ghrelin 24-29GC or 24-29C Coupled to Qβ VLP in a Diet Induced Animal Model of Obesity

Adult, male or female, C57BL/6 mice with comparable starting weights are vaccinated, as described in EXAMPLE 10 with either ghrelin 24-30C, 24-29GC or 24-29C coupled to Qβ VLP, obtained in EXAMPLE 11. As a control, mice are immunized with Qβ VLP alone or injected with PBS. Mice are subsequently boosted if ghrelin-specific antibody titers significantly decline during the experiment. All mice are placed on a high fat diet (35% fat by weight, 60% as energy) to facilitate the development of diet-induced obesity. Food and water is administered ad libitum. Body weights are monitored at regular intervals.

### Example 15

### Efficacy Experiments with Ghrelin 24-31GC, Ghrelin 24-31C, Ghrelin 24-30GC, Ghrelin 24-30C, Ghrelin 24-29GC or 24-29C Coupled to Qβ VLP in a Genetic Animal Model of Obesity

Adult male or female, C57BL/6 ob/ob mice are vaccinated as described in EXAMPLE 12 with either murine ghrelin 24-31GC, ghrelin 24-31C, ghrelin 24-30GC, ghrelin 24-30C, ghrelin 24-29GC or ghrelin 24-29C coupled to prior art Qβ VLP, obtained in EXAMPLE 9. As a control, mice are immunized with Qβ VLP or injected with PBS. Mice are subsequently boosted if ghrelin-specific antibody titers significantly decline over the period of the experiment. Mice are fed a standard diet (consisting of 4-10% fat by weight), ad libitum, and have free access to water. Body weights are monitored at regular intervals.

### Example 16

### Efficacy Experiments with Ghrelin 24-31 GC, Ghrelin 24-31C, Ghrelin 24-30GC, Ghrelin 24-30C, Ghrelin 24-29GC or Ghrelin 24-29C Coupled to Qβ VLP in a Therapeutic Diet-Induced Animal Model of Obesity

Adult male or female, C57BL/6 mice are fed a high fat diet, ad libitum, for approximately 17-24 weeks or until they have become obese (weights>45 g). Mice are then grouped such that the distribution of the starting weights and the average starting weights are similar for all groups.

Mice are vaccinated as described in EXAMPLE 10, with either murine ghrelin 24-31GC, ghrelin 24-31C, ghrelin 24-30GC, ghrelin 24-30C, ghrelin 24-29GC or ghrelin 24-29C coupled to Qβ VLP, obtained in EXAMPLE 9. As a control mice are immunized with Qβ VLP or injected with PBS. Mice are further boosted if ghrelin-specific antibody titers start to decline. Mice are bled retro-orbitally on day 0, 14, 28, 42, 56, 70 and then at monthly intervals. The sera are analyzed for ghrelin-specific antibodies by ELISA as described in EXAMPLE 11. Body weights are monitored at regular intervals.

### Example 17

### Blocking Migration of Exogenous Radioactive Ghrelin to the Brain with Ghrelin 24-31GC Coupled to Qβ VLP, in an In Vivo Animal Model

Adult female, C57BL/6 mice (3 per group) were vaccinated, as described in EXAMPLE 10, with ghrelin 24-31GC coupled to Qβ VLP, obtained in EXAMPLE 9. As a control, mice were injected with Qβ VLP. A further immunization was administered on day 174 and 14 days later all mice were challenged intravenously with 10 ng of iodinated, serine-octanoylated murine ghrelin (I¹²⁵-Ghrelin). Thirty minutes after challenge, mice were sacrificed and serum and brain tissue collected. The level of radioactivity was measured by scintillation count and the amount of I¹²⁵-Ghrelin present in the serum and brain of individual mice was calculated.

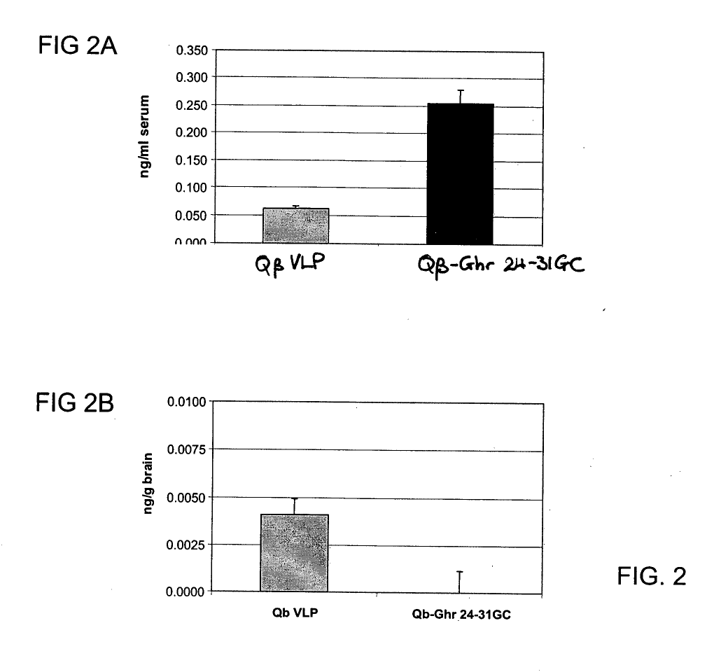

FIG. 2 shows a marked reduction in I¹²⁵-Ghrelin in the brain and an increased amount of I¹²⁵-Ghrelin in the serum of mice immunized with ghrelin 24-31GC coupled to Qβ VLP, compared to Qβ VLP immunized control mice. Despite administering a 60-fold excess of I¹²⁵-Ghrelin, compared to physiological blood ghrelin concentrations, ghrelin-specific antibodies were able to bind and prevent the passage of I¹²⁵-Ghrelin from the blood to the brain. This result clearly demonstrates that a ghrelin-VLP conjugate is able to sequester ghrelin in the serum and hence, block it from exerting its effect in the brain.

### Example 18

### Blocking Ghrelin-Induced Growth Hormone Secretion with Ghrelin 24-31 GC Coupled to Qβ VLP, in an In Vivo Animal Model

Adult female, C57BL/6 mice (5 per group) were vaccinated, as described in EXAMPLE 10, with ghrelin 24-31GC coupled to Qβ VLP, obtained in EXAMPLE 9. As a control, mice were injected with Qβ VLP. Approximately 6 weeks later (day 80), mice were fasted for 48 hours and then challenged intravenously with 10 μg of serine-octanoylated murine ghrelin. Five minutes after challenge, mice were bled retro-orbitally and serum collected. Growth hormone levels in the serum were measured by a growth hormone specific ELISA (Rat Growth hormone Biotrak assay, Amersham).

TABLE 3 shows a marked reduction in ghrelin-induced growth hormone release in mice immunized with ghrelin 24-31GC coupled to Qβ VLP, compared to Qβ VLP immunized control mice. Ghrelin-specific antibodies were able to bind and sequester the exogenously administered ghrelin in the serum, hence, blocking its effect on growth hormone release. This result clearly demonstrates that a ghrelin-VLP conjugate is able to prevent ghrelin-induced growth hormone release.

TABLE 3
Average growth hormone levels, 5 minutes after challenge with
10 μg serine-octanoylated ghrelin, in mice immunized with
Qβ-Ghrelin 24-31GC or Qβ VLP on day 0, 14, 28 and 42.

| | Time after ghrelin challenge (±SEM) | |
|---|---|---|
| | t = 0 min | t = 5 min |
| Qb-Ghrelin 24-31GC | 91 ± 10 | 146 ± 17 |
| Qb VLP | 159 ± 44 | 493 ± 195 |

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

1. A modified virus like particle (VLP) comprising:
(a) a virus like particle (VLP), and
(b) at least one ghrelin-peptide,
wherein said ghrelin-peptide consists of a peptide with a length of 6 or 8 amino acid residues, which peptide is homologous to or identical with SEQ ID NO:1 or SEQ ID NO: 3 and wherein (a) and (b) are linked with one another.

2. The modified VLP of claim 1 wherein said ghrelin-peptide is selected from SEQ ID NO:1 or SEQ ID NO: 3, and preferably wherein said ghrelin-peptide is SEQ ID NO: 3.

3. The modified VLP of claim 1, wherein said ghrelin-peptide differs at only 1 position from SEQ ID NO:1 or SEQ ID NO: 3, and wherein preferably said difference is an amino acid substitution, and even more preferably a conservative amino acid substitution.

4. The modified VLP of claim 1, whrein the ghrelin-peptide is a mammalian ghrelin-peptide, in particular a ghrelin-peptide from human, dog, cat, cow, sheep, horse, mouse or rat.

5. The modified VLP of claim 1, wherein said ghrelin-peptide does not contain a n-octanoyl-modification, and wherein preferably said ghrelin-peptide does not contain a n-octanoyl-modification at position 3 of SEQ ID NO:1 or SEQ ID NO: 3, and wherein even more preferably said ghrelin-peptide does not contain a n-octanoyl-modification at position 3 of SEQ ID NO: 3.

6. The modified VLP of of claim 1, wherein said virus-like particle comprises recombinant proteins selected from the group consisting of:
(a) recombinant proteins of RNA-phages;
(b) recombinant proteins of bacteriophages;
(c) recombinant proteins of Sindbis virus;
(d) recombinant proteins of Rotavirus;
(e) recombinant proteins of Foot-and-Mouth-Disease virus;
(f) recombinant proteins of Retrovirus;
(g) recombinant proteins of Norwalk virus;
(h) recombinant proteins of Alphavirus;
(i) recombinant proteins of human Papilloma virus;
(j) recombinant proteins of Polyoma virus;
(k) recombinant proteins of measles virus;
(l) recombinant proteins of Hepatitis B virus;
(m) recombinant proteins of Ty; and
(n) fragments of any recombinant proteins of (a) to (m) being capable of assembling into a VLP,

7. The modified VLP of claim 1, wherein said VLP comprises, or alternatively consists of, recombinant proteins, or fragments thereof being capable of assembling into a VLP, of a RNA-phage, and wherein said RNA-phage is Qβ, fr, AP205 or GA.

8. The modified VLP of claim 1, wherein the VLP (a) is linked with the ghrelin-peptide (b) through at least one covalent bond.

9. The modified VLP of claim 1, wherein said ghrelin-peptide is fused to said VLP.

10. The modified VLP of claim 1, wherein the VLP (a) is linked with the ghrelin-peptide (b) through at least one non-peptide bond.

11. The modified VLP of claim 1 further comprising an amino acid linker, and wherein preferably said amino acid linker is selected from the group consisting of:

| | |
|---|---|
| (a) | GGC; |
| (b) | GGC-CONH2; |
| (c) | GC; |
| (d) | GC-CONH2; |
| (e) | C; |
| | and |
| (f) | C-CONH2. |

12. The modified VLP of claim 1, wherein said ghrelin-peptide is linked via its C-terminus to the VLP.

13. The modified VLP of claim 1, wherein said VLP particle comprises least one first attachment site; and wherein said at least one ghrelin-peptide comprises at least one second attachment site being selected from the group consisting of (i) an attachment site not naturally occurring with said ghrelin-peptide; and (ii) an attachment site naturally occurring with said ghrelin-peptide, and wherein said second attachment site is capable of association to said first attachment site, preferably to form an ordered and repetitive antigen array.

14. The modified VLP of claim 13, wherein said ghrelin-peptide with said added at least one second attachment site comprises, or alternatively consists of an amino acid sequence selected from the group consisting of

| | | | |
|---|---|---|---|
| (a) | Ghrel24-31GC: | GSSFLSPEGC; | (SEQ ID NO:50) |
| or | | | |
| (b) | Ghrel24-31C: | GSSFLSPEC. | (SEQ ID NO:51) |

15. The modified VLP of claim 13, wherein said first attachment site comprises, or preferably is, an amino group, and wherein even further preferably said first attachment site is an amino group of a lysine residue.

16. The modified VLP of any of claims 13, wherein said second attachment site comprises, or preferably is, a sulfhydryl group, and wherein even further preferably said second attachment site is a sulfhydryl group of a cysteine residue.

17. A composition comprising a modified VLP of claim 1.

18. A pharmaceutical composition comprising:
(a) The modified VLP of claim 1; and
(b) an acceptable pharmaceutical carrier.

19. A vaccine composition comprising a modified VLP of claim 1.

20. The vaccine composition of claim 19, wherein said vaccine composition is devoid of an adjuvant.

21. A process for producing the modified VLP of claim 1:
(a) providing a VLP with at least one first attachment site;
(b) providing at least one ghrelin-peptide with at least one second attachment site
wherein said second attachment site is capable of association to said first attachment site; and
(c) combining said VLP and said ghrelin-peptide to produce a modified VLP, wherein said ghrelin-peptide and said VLP interact through said association.

22. A method of immunization comprising administering a modified VLP of claim 1 to an animal or human.

23. The method of immunization of claim 22, wherein (i) said animal is a human, and wherein said ghrelin-peptide is a human ghrelin-peptide; (ii) said animal is of feline origin, and wherein said ghrelin-peptide is a feline ghrelin-peptide; or (iii) said animal is of canine origin, and wherein said ghreline peptide is a canine ghreline peptide.


SEQUENCE LISTING (74 sequences)

SEQ ID NO: 1 - protein, 6 aa, homo sapiens
GSSFLS

SEQ ID NO: 2 - protein, 7 aa, homo sapiens
GSSFLSP

SEQ ID NO: 3 - protein, 8 aa, homo sapiens
GSSFLSPE

SEQ ID NO: 4 - protein, 132 aa, Bacteriophage Q-beta
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 5 - protein, 329 aa, Bacteriophage Q-beta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYWTLLIAGGGSGSKPDPVIPDPPIDPPPGTGKYTCPFAIWSLEEVYEPPTKNRPWPIYNAVELQPREFDVALKDLLGNTKWRDWDSRLSYTTFRGCRGNGYIDLDATYLATDQAMRDQKYDIREGKKPGAFGNIERFIYLKSINAYCSLSDIAAYHADGVIVGFWRDPSSGGAIPFDFTKFDKTKCPIQAVIVVPRA

SEQ ID NO: 6 - protein, 129 aa, Bacteriophage R17
ASNFTQFVLVNDGGTGNVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 7 - protein, 130 aa, Bacteriophage fr
MASNFEEFVLVDNGGTGDVKVAPSNFANGVAEWISSNSRSQAYKVTCSVRQSSANNRKYTVKVEVPKVATQVQGGVELPVAAWRSYMNMELTIPVFATNDDCALIVKALQGTFKTGNPIATAIAANSGIY

SEQ ID NO: 8 - protein, 130 aa, Bacteriophage GA
MATLRSFVLVDNGGTGNVTVVPVSNANGVAEWLSNNSRSQAYRVTASYRASGADKRKYAIKLEVPKIVTQVVNGVELPGSAWKAYASIDLTIPIFAATDDVTVISKSLAGLFKVGNPIAEAISSQSGFYA

SEQ ID NO: 9 - protein, 132 aa, Bacteriophage SP
MAKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAY

SEQ ID NO: 10 - protein, 329 aa, Bacteriophage SP
AKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAYWAALLVASSGGGDNPSDPDVPVVPDVKPPDGTGRYKCPFACYRLGSIYEVGKEGSPDIYERGDEVSVTFDYALEDFLGNTNWRNWDQRLSDYDIANRRRCRGNGYIDLDATAMQSDDFVLSGRYGVRKVKFPGAFGSIKYLLNIQGDAWLDLSEVTAYRSYGMVIGFWTDSKSPQLPTDFTQFNSANCPVQTVIIIPS

SEQ ID NO: 11 - protein, 130 aa, Bacteriophage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 12 - protein, 133 aa, Bacteriophage M11
MAKLQAITLSGIGKKGDVTLDLNPRGVNPTNGVAALSEAGAVPALEKRVTISVSQPSRNRKNYKVQVKIQNPTSCTASGTCDPSVTRSAYSDVTFSFTQYSTVEERALVRTELQALLADPMLVNAIDNLNPAY

SEQ ID NO: 13 - protein, 133 aa, Bacteriophage MX1
MAKLQAITLSGIGKNGDVTLNLNPRGVNPTNGVAALSEAGAVPALEKRVTISVSQPSRNRKNYKVQVKIQNPTSCTASGTCDPSVTRSAYADVTFSFTQYSTDEERALVRTELKALLADPMLIDAIDNLNPAY

SEQ ID NO: 14 - protein, 330 aa, Bacteriophage NL95
MAKLNKVTLTGIGKAGNQTLTLTPRGVNPTNGVASLSEAGAVPALEKRVTVSVAQPSRNRKNYKVQIKLQNPTACTKDACDPSVTRSGSRDVTLSFTSYSTERERALIRTELAALLKDDLIVDAIDNLNPAYWAALLAASPGGGNNPYPGVPDSPNVKPPGGTGTYRCPFACYRRGELITEAKDGACALYACGSEALVEFEYALEDFLGNEFWRNWDGRLSKYDIETHRRCRGNGYVDLDASVMQSDEYVLSGAYDVVKMQPPGTFDSPRYYLHLMDGIYVDLAEVTAYRSYGMVIGFWTDSKSPQLPTDFTRFNRHNCPVQTVIVIPSL

SEQ ID NO: 15 - protein, 129 aa, Bacteriophage f2
ASNFTQFVLVNDGGTGNVTVAPSNFANGVAEWISSNSRSQAYKVTCSVRQSSAQNRKYTIKVEVPKVATQTVGGVELPVAAWRSYLNLELTIPIFATNSDCELIVKAMQGLLKDGNPIPSAIAANSGIY

SEQ ID NO: 16 - protein, 128 aa, Bacteriophage PP7
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 17 - protein, 132 aa, Artificial Sequence
  Bacteriophage Qbeta 240 mutant
AKLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 18 - protein, 132 aa, Artificial Sequence
  Bacteriophage Q-beta 243 mutant
AKLETVTLGKIGKDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 19 - protein, 132 aa, Artificial Sequence
  Bacteriophage Q-beta 250 mutant
ARLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 20 - protein, 132 aa, Artificial Sequence
  Bacteriophage Q-beta 251 mutant
AKLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 21 - protein, 132 aa, Artificial Sequence
  Bacteriophage Q-beta 259 mutant
ARLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 22 - protein, 185 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 23 - protein, 212 aa, Hepatitis B virus
MQLFHLCLIISCSCPTVQASKLCLGWLWGMDIDPYKEFGATVELLSFLPSDFFPSVRDLLDTASALYREALESPEHCSPHHTALRQAILCWGDLMNLATWVGGNLEDPVSRDLVVGYVNTTVGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 24 - protein, 188 aa, Hepatitis B virus
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSTNC

SEQ ID NO: 25 - protein, 185 aa, Hepatitis B virus; identical to SEQ ID NO: 22

SEQ ID NO: 26 - protein, 188 aa, Hepatitis B virus
MDIDPYKEFGATVELLSFLPSDFFPSVRDLLDTAAALYRDALESPEHCSPHHTALRQAILCWGDLMTLATWVGTNLEDGGKGGSRDLVVSYVNTNVGLKFRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 27 - DNA, 3635 bp, Artificial Sequence
  plasmid pAP283-58
cgagctcgcccctggcttatcgaaattaatacgactcactatagggagaccggaattcgagctcgcccggggatcctctagaattttctgcgcacccatcccgggtggcgcccaaagtgaggaaaatcacatggcaaataagccaatgcaaccgatcacatctacagcaaataaaattgtgtggtcggatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgcttaagcttgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgttccgcaccccggaaatggtcaccgaaccaatcagcagggtcatcgctagccagatcctctacgccggacgcatcgtggccggcatcaccggcgcacacagtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgcttcaacggcctcaacctactactgggctgcttcctaatgcaggagtcgcataagggagagcgtcgatatggtgcactctcagtacaatctgctctgatgccgcatagttaagccaactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgcgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgacgagcgcagcgagtcagtgagcgaggaagcggaagagcgcccaatacgcaaaccgcctctccccgcgcgttggccgattcattaatgcagctgtggtgtcatggtcggtgatcgccagggtgccgacgcgcatctcgactgcatggtgcaccaatgcttctggcgtcaggcagccatcggaagctgtggtatggccgtgcaggtcgtaaatcactgcataattcgtgtcgctcaaggcgcactcccgttctggataatgttttttgcgccgacatcataacggttctggcaaatattctgaaatgagctgttgacaattaatcatcgaactagttaactagtacgcaagttcacgtaaaaagggtatcgcggaatt

SEQ ID NO: 28 - protein, 131 aa, Bacteriophage AP205
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 29 - protein, 131 aa, Artificial Sequence
  AP205 coat protein
MANKTMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 30 - DNA, 3607 bp, Artificial Sequence
  plasmid pAP281-32
cgagctcgcccctggcttatcgaaattaatacgactcactatagggagaccggaattcgagctcgcccggggatcctctagattaacccaacgcgtaggagtcaggccatggcaaataagacaatgcaaccgatcacatctacagcaaataaaattgtgtggtcggatccaactcgtttatcaactacattttcagcaagtctgttacgccaacgtgttaaagttggtatagccgaactgaataatgtttcaggtcaatatgtatctgtttataagcgtcctgcacctaaaccgaaggtcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgcttaagcttgtattctatagtgtcacctaaatcgtatgtgtatgatacataaggttatgtattaatggtagccgcgttctaacgacaatatgtacaagcctaattgtgtagcatctggcttactgaagcagaccctatcatctctctcgtaaactgccgtcagagtcggttgggttggacagacctctgagtttctggtaacgccgttccgcaccccggaaatggtcaccgaaccattcagcagggtcatcgctagccagatcctctacgccggacgcatcgtggcccgcatcaccggcgccacaggtgcggtgctggcgcctatatcgccgacatcaccgatggggaagatcgggctcgccacttcgggctcatgatcgctggtttccgcctgggtatggtggcaggccccgtggcccgggggactgttgggcgccatctccttgcatgcaccattccttgcggcggcggtgctcaacggcctcaacctactactgggctgcttcctaatgcaggagtcgcataagggagagcgtcgatatggtgcactctcagtacaatctgctctgatgccgcatagttaagccaactccgctatcgctacgtgactgggtcatggctgcgccccgacacccgccaacacccgctgacgcgccctgacgggcttgtctgcttccggcatccgcttacagacaagctgtgaccgtctccgggagctgcatgtgtcagaggttttcaccgtcatcaccgaaacgcgcgaggcagcttgaagacgaaagggcctcgtgatacgcctatttttataggttaatgtcatgataataatggtttcttagacgtcaggtggcacttttcggggaaatgtgcgcggaccccctattggtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgtttttcaatgatgagcacttttaaagttctgctatgtgtcgcggtattatcccgtattgacgccgggcaagagcaactcggtcgccgcatacactattctcagaatgacttggtggtacctaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtacgaacggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcggtcagaccccgtagaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgataggtaccggataaggcgcagcggtcgggctgaacggggggttcgtgcacacagcccagcttggagcgaacgacctacaccgaactgagatacctacagcgcgagcattgagaaagcgccacgcttcccgaagggagaaaggcggacaggtatccggtaagcggcagggtcggaacaagagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcctttggctggccttttgctcacatgttctttcctgcgttatcccctgattctgtggataaccgtattaccgcctttgagtgagctgataccgctcgccgcagccgaacgaccgacggcgcagcgagtcagtgagcgaggaagcggaagagcgcccaatacgcaaaccgcctctccccgcgcgttggccgattcattaatgcagctgtggtgtcatggtcggtgatcgccagggtgccgacgcgcatctcgactgcatggtgcaccaatgcttctggcgtcaggcagccatcggaagctgtggtatggccgtgcaggtcgtaaatcactgcataattcgtgtcgctcaaggcgcactcccgttctggataatgttttttgcggcgacatcataacggttctggcaaatattctgaaatgagctggtgacaattaatcatcgaactagttaactagtacgcaagttcacgtaaaaagggtatcgcggaatt

SEQ ID NO: 31 - protein, 28 aa, Homo sapiens
GSSFLSPEHQRVQQRKESKKPPAKLQPR

SEQ ID NO: 32 - protein, 28 aa, Mus musculus
GSSFLSPEHQKAQQRKESKKPPAKLQPR

SEQ ID NO: 33 - protein, 5 aa, Artificial Sequence
  Linker
GGKGG

SEQ ID NO: 34 - protein, 3 aa, Artificial Sequence
  N-terminal glycine linker
GCG

SEQ ID NO: 35 - protein, 9 aa, Artificial Sequence
  N terminal glycine serine linkers
GCGSGGGGS

SEQ ID NO: 36 - protein, 3 aa, Artificial Sequence
  C-terminal glycine linker
GCG

SEQ ID NO: 37 - protein, 10 aa, Artificial Sequence
  C terminal glycine serine linkers
GSGGGGSGCG

SEQ ID NO: 38 - protein, 5 aa, Artificial Sequence
  Glycine serine linker
GGGGS

SEQ ID NO: 39 - protein, 10 aa, Artificial Sequence
  N-terminal gamma1
CGDKTHTSPP

SEQ ID NO: 40 - protein, 10 aa, Artificial Sequence
  C-terminal gamma 1
DKTHTSPPCG

SEQ ID NO: 41 - protein, 17 aa, Artificial Sequence
  N-terminal gamma 3
CGGPKPSTPPGSSGGAP

SEQ ID NO: 42 - protein, 18 aa, Artificial Sequence
  C-terminal gamma 3
PKPSTPPGSSGGAPGGCG

SEQ ID NO: 43 - protein, 6 aa, Artificial Sequence
  N-terminal glycine linker
GCGGGG

SEQ ID NO: 44 - protein, 6 aa, Artificial Sequence
  C-terminal glycine linker
GGGGCG

SEQ ID NO: 45 - protein, 6 aa, Artificial Sequence
  C-terminal glycine-lysine linker
GGKKGC

SEQ ID NO: 46 - protein, 6 aa, Artificial Sequence
  N-terminal glycine-lysine linker
CGKKGG

SEQ ID NO: 47 - protein, 4 aa, Artificial Sequence
  C.terminal linker
GGCG

SEQ ID NO: 48 - DNA, 57 bp, Artificial Sequence
  AP205 ribosomal binding site
tctagaattttctgcgcacccatcccgggtggcgcccaaagtgaggaaaatcacatg

SEQ ID NO: 49 - DNA, 35 bp, Artificial Sequence
  Shine-Dalgarno sequence of vector pQb185
tctagattaacccaacgcgtaggagtcaggccatg

SEQ ID NO: 50 - protein, 10 aa, Artificial Sequence
  Ghrelin peptide mutant 24-31GC
GSSFLSPEGC

SEQ ID NO: 51 - protein, 9 aa, Artificial Sequence
  Ghrelin peptide mutant 24-31C
GSSFLSPEC

SEQ ID NO: 52 - protein, 9 aa, Artificial Sequence
  Ghrelin peptide mutant 24-30GC
GSSFLSPGC

SEQ ID NO: 53 - protein, 8 aa, Artificial Sequence
  Ghrelin peptide mutant 24-30C
GSSFLSPC

SEQ ID NO: 54 - protein, 8 aa, Artificial Sequence
  Ghrelin peptide mutant 24-29GC
GSSFLSGC

SEQ ID NO: 55 - protein, 7 aa, Artificial Sequence
  Ghrelin peptide mutant 24-29C
GSSFLSC

SEQ ID NO: 56 - DNA, 31 bp, Artificial Sequence
  EcoRIHBcAg(s) primer
ccggaattcatggacattgacccttataaag

SEQ ID NO: 57 - DNA, 51 bp, Artificial Sequence
  Lys-HBcAg(as) primer
cctagagccacctttgccaccatcttctaaattagtacccacccaggtagc

SEQ ID NO: 58 - DNA, 48 bp, Artificial Sequence
  Lys-HBcAg(s) primer
gaagatggtggcaaaggtggctctagggacctagtagtcagttatgtc

SEQ ID NO: 59 - DNA, 38 bp, Artificial Sequence
  HBcAgwtHindIIII primer
cgcgtcccaagcttctaacattgagattcccgagattg

SEQ ID NO: 60 - protein, 10 aa, Artificial Sequence
  epitope CeH3
VNLTWSRASG

SEQ ID NO: 61 - DNA, 51 bp, Artificial Sequence
  CeH3fwd primer
gttaacttgacctggtctcgtgcttctggtgcatccagggatctagtagtc

SEQ ID NO: 62 - protein, 17 aa, Artificial Sequence
  CeH3fwd primer
VNLTWSRASGASRDLVV

SEQ ID NO: 63 - DNA, 51 bp, Artificial Sequence
  CeH3rev primer
accagaagcacgagaccaggtcaagttaacatcttccaaattattacccac

SEQ ID NO: 64 - protein, 7 aa, Artificial Sequence
  CeH3rev primer peptide
DELNNGV

SEQ ID NO: 65 - DNA, 31 bp, Artificial Sequence; identical to SEQ ID NO: 56
  HBcAg-wt EcoRI fwd primer

SEQ ID NO: 66 - DNA, 38 bp, Artificial Sequence; identical to SEQ ID NO: 59
  HBcAg-wt Hind III rev primer

SEQ ID NO: 67 - DNA, 28 bp, Artificial Sequence
  primer p1.44
aaccatggcaaataagccaatgcaaccg

SEQ ID NO: 68 - DNA, 30 bp, Artificial Sequence
  primer p1.45
aatctagaattttctgcgcacccatcccgg

SEQ ID NO: 69 - DNA, 31 bp, Artificial Sequence
  primer p1.46
aaaagcttaagcagtagtatcagacgatacg

SEQ ID NO: 70 - DNA, 43 bp, Artificial Sequence
  primer p1.47
gagtgatccaactcgtttatcaactacattttcagcaagtctg

SEQ ID NO: 71 - DNA, 43 bp, Artificial Sequence
  primer p1.48
cagacttgctgaaaatgtagttgataaacgagttggatcactc

SEQ ID NO: 72 - protein, 117 aa, Homo sapiens
MPSPGTVCSLLLLGMLWLDLAMAGSSFLSPEHQRVQQRKESKKPPAKLQPRALAGWLRPEDGGQAEGAEDELEVRFNAPFDVGIKLSGVQYQQHSQALGKFLQDILWEEAKEAPADK

SEQ ID NO: 73 - protein, 117 aa, Canis familiaris
MPSPGTVCSLLLLGMLWLDLAMAGSSFLSPEHQKLQQRKESKKPPAKLQPRALAGWLRPEDGGQAEGAEDELEVRFNAPFDVGIKLSGVQYQQHSQALGKFLQDILWEEAKEAPADK

SEQ ID NO: 74 - protein, 117 aa, Mus musculus
MPSPGTVCSLLLLGMLWLDLAMAGSSFLSPEHQKAQQRKESKKPPAKLQPRALAGWLRPEDGGQAEGAEDELEVRFNAPFDVGIKLSGVQYQQHSQALGKFLQDILWEEAKEAPADK